SEQUENCE LISTING (34 sequences)

SEQ ID NO: 1 - protein, 467 aa, Homo sapiens
MWRCPLGLLLLLPLAGHLALGAQQGRGRRELAPGLHLRGIRDAGGRYCQEQDLCCRGRADDCALPYLGAICYCDLFCNRTVSDCCPDFWDFCLGVPPPFPPIQGCMHGGRIYPVLGTYWDNCNRCTCQENRQWQCDQEPCLVDPDMIKAINQGNYGWQAGNHSAFWGMTLDEGIRYRLGTIRPSSSVMNMHEIYTVLNPGEVLPTAFEASEKWPNLIHEPLDQGNCAGSWAFSTAAVASDRVSIHSLGHMTPVLSPQNLLSCDTHQQQGCRGGRLDGAWWFLRRRGVVSDHCYPFSGRERDEAGPAPPCMMHSRAMGRGKRQATAHCPNSYVNNNDIYQVTPVYRLGSNDKEIMKELMENGPVQALMEVHEDFFLYKGGIYSHTPVSLGRPERYRRHGTHSVKITGWGEETLPDGRTLKYWTAANSWGPAWGERGHFRIVRGVNECDIESFVLGVWGRVGMEDMGHH

SEQ ID NO: 2 - protein, 467 aa, Canus lupus
MWRCPLGLLLLLLLAGESALGARRGRGRRELAPGLHLRGIRDAGGRYCQEQDLCCRGRADDCALPYLGATCYCDLFCNRTVSDCCPDFWDFCLGVPPPFPPIQGCTHGSRIYPVLGTYWDNCNRCTCQEQGRWECDQEPCLVDQDMINAINQGNYGWRAGNHSAFWGMTLDEGIRYRLGTIRPSSSVTNMNEIHTVLRPGEVLPTAFEAAEKWPNLIHEPLDQGNCAGSWAFSTAAVASDRVSIHSLGHMTPVLSPQNLLSCDTHNQQGCRGGRLDGAWWFLRRRGVVSDHCYPFVGREQDEAGPAPRCMMHSRAMGRGKRQATARCPSSHVHANDIYQVTPAYRLGTNEKEIMKELMENGPVQALMEVHEDFFLYQGGIYSHTPVSLGRPERYRRHGTHSVKITGWGEETLPDGRTLKYWTAANSWGPAWGERGHFRIVRGANECDIESFVLGVWGRVGMEDMGHR

SEQ ID NO: 3 - protein, 467 aa, Equus caballus
MWRCPLGLLLLLLLAGELALGARRGRGRRELAPALHLRGIRDAGGRYCQEQDLCCRGRADECALPYLGVTCYCDLFCNRTVSDCCPDFWDFCLGVPPPFPPVQGCVHRGRVYPVLGTYWDNCNRCTCQEKGQWECDQEPCLVDQDMINAINQGNYGWRAGNHSAFWGMTLDEGIRYRLGTIRPSSSVTSMNEIHTVLGPGEVLPTAFEASEKWPNLIHEPLDQGNCAGSWAFSTAAVASDRVSIHSLGHMTPVLSPQNLLSCDTHNQQGCRGGHLDGAWWFLRRRGVVSDHCYPFSGRERDEAGPAPRCMMHSRAMGRGKRQATAHCPNSRVHTNDIYQVTPAYRLGSSEKEIMKELMENGPVQALMEVHEDFFLYQGGVYSHTPVSHGRPERYRRHGTHSVKITGWGEETLPDGRTLKYWTAANSWGPAWGERGHFRIVRGANECDIESFVLGVWGRVGMEDMGHH

SEQ ID NO: 4 - protein, 466 aa, Mus musculus
MWGCWLGLLLLLLAGQAALEARRSRWRRELAPGLHLRGIRDAGGRYCQEQDMCCRGRADECALPYLGATCYCDLFCNRTVSDCCPDFWDFCLGIPPPFPPVQGCMHGGRIYPVFGTYWDNCNRCTCHEGGHWECDQEPCLVDPDMIKAINRGNYGWQAGNHSAFWGMTLDEGIRYRLGTIRPSSTVMNMNEIYTVLGQGEVLPTAFEASEKWPNLIHEPLDQGNCAGSWAFSTAAVASDRVSIHSLGHMTPILSPQNLLSCDTHHQQGCRGGRLDGAWWFLRRRGVVSDNCYPFSGREQNEASPTPRCMMHSRAMGRGKRQATSRCPNGQVDSNDIYQVTPAYRLGSDEKEIMKELMENGPVQALMEVHEDFFLYQRGIYSHTPVSQGRPEQYRRHGTHSVKITGWGEETLPDGRTIKYWTAANSWGPWWGERGHFRIVRGTNECDIETFVLGVWGRVGMEDMGHH

SEQ ID NO: 5 - protein, 23 aa, Artificial Sequence
  Forward qRT-PCR Primer for hTINAGL1
TCTTCCTCGGTCATGAACATGCA

SEQ ID NO: 6 - protein, 23 aa, Artificial Sequence
  Reverse qRT-PCR Primer for hTINAGL1
TTGCCTTGGTCAAGAGGCTCATG

SEQ ID NO: 7 - protein, 20 aa, Artificial Sequence
  Forward qRT-PCR Primer for hGAPDH
TGCACCACCAACTGCTTAGC

SEQ ID NO: 8 - protein, 21 aa, Artificial Sequence
  Reverse qRT-PCR Primer for hGAPDH
GGCATGGACTGTGGTCATGAG

SEQ ID NO: 9 - protein, 21 aa, Artificial Sequence
  Forward qRT-PCR Primer for mTinagl1
TCTTTCTCCGTGAGTTGCAGT

SEQ ID NO: 10 - protein, 21 aa, Artificial Sequence
  Reverseq RT-PCR Primer for mTinagl1
CATGGTGCCTCCTGGAGTAGC

SEQ ID NO: 11 - protein, 23 aa, Artificial Sequence
  Forward qRT-PCR Primer for mGapdh
TCCCACTCTTCCACCTTCGATGC

SEQ ID NO: 12 - protein, 24 aa, Artificial Sequence
  Reverse qRT-PCR Primer for mGapdh
GGGTCTGGGATGGAAATTGTGAGG

SEQ ID NO: 13 - protein, 20 aa, Artificial Sequence
  Forward PCR Primer for TINAGL1
TGTGGCGATGTCCACTGGGG

SEQ ID NO: 14 - protein, 22 aa, Artificial Sequence
  Reverse PCR Primer for TINAGL1
GTGATGACCCATGTCCTCCATG

SEQ ID NO: 15 - protein, 24 aa, Artificial Sequence
  Forward PCR Primer for Wild type Integrin subunit
ATGAATTTACAACCAATTTTCTGG

SEQ ID NO: 16 - protein, 25 aa, Artificial Sequence
  Reverse PCR Primer for Wild type Integrin subunit
TCATTTTCCCTCATACTTCGGATTG

SEQ ID NO: 17 - protein, 24 aa, Artificial Sequence
  Forward PCR Primer for Mutant Integrin subunit 1
ATGAATTTACAACCAATTTTCTGG

SEQ ID NO: 18 - protein, 23 aa, Artificial Sequence
  Reverse PCR Primer for Mutant Integrin subunit 1
TTCAGCTCTCTTGAATTTTAATG

SEQ ID NO: 19 - protein, 25 aa, Artificial Sequence
  Forward PCR Primer for Mutant Integrin subunit 1
ATATCTGGAAATTTGGATTCTCCAG

SEQ ID NO: 20 - protein, 25 aa, Artificial Sequence
  Reverse PCR Primer for Mutant Integrin subunit 1
TCATTTTCCCTCATACTTCGGATTG

SEQ ID NO: 21 - protein, 20 aa, Artificial Sequence
  Forward PCR Primer for EGFR
ATGCGACCCTCCGGGACGGC

SEQ ID NO: 22 - protein, 20 aa, Artificial Sequence
  Reverse PCR Primer for EGFR
TGCTCCAATAAATTCACTGC

SEQ ID NO: 23 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 24 - protein, 475 aa, Homo sapiens
MWRCPLGLLLLLPLAGHLALGAQQGRGRRELAPGLHLRGIRDAGGRYCQEQDLCCRGRADDCALPYLGAICYCDLFCNRTVSDCCPDFWDFCLGVPPPFPPIQGCMHGGRIYPVLGTYWDNCNRCTCQENRQWQCDQEPCLVDPDMIKAINQGNYGWQAGNHSAFWGMTLDEGIRYRLGTIRPSSSVMNMHEIYTVLNPGEVLPTAFEASEKWPNLIHEPLDQGNCAGSWAFSTAAVASDRVSIHSLGHMTPVLSPQNLLSCDTHQQQGCRGGRLDGAWWFLRRRGVVSDHCYPFSGRERDEAGPAPPCMMHSRAMGRGKRQATAHCPNSYVNNNDIYQVTPVYRLGSNDKEIMKELMENGPVQALMEVHEDFFLYKGGIYSHTPVSLGRPERYRRHGTHSVKITGWGEETLPDGRTLKYWTAANSWGPAWGERGHFRIVRGVNECDIESFVLGVWGRVGMEDMGHHVDHHHHHH

SEQ ID NO: 25 - protein, 471 aa, Danio rerio
MLRLWVLAAVSVLLLLSEGGMTARTKRELAGPLHLRGIRDPFGSYCQRRGGCCPGRNDQCTVPYLDTICYCDLFCNRTVSDCCPDFWGHCLGTTPPYPPSSCERNGHRFPSGSTYKENCNLCTCGQNGRWECEQHACLIEDDMIQEINRRDYGWRAANYSQFWGMTLDEGLRFRLGTKRPTRTIMNMNEMQMNMNGNDHLPSYFNAVDKWPGKIHEPLDQGNCNASWAFSTAAVASDRISIQSMGHMTPQLSPQNLISCDTRHQDGCAGGRIDGAWWFMRRRGVVTQDCYPFSPPEQSAVEVARCMMQSRAVGRGKRQATAHCPNSHSYHNDIYQSTPPYRLSTNENEIMKEIMDNGPVQAIMEVHEDFFVYKSGIFRHTDVNYHKPSQYRKHATHSVRITGWGEERDYSGRTRKYWIGANSWGKNWGEDGYFRIARGVNECDIETFVIGVWGRVTMEDMHNHHHHHGRRK

SEQ ID NO: 26 - protein, 467 aa, Rattus norvegicus
MWGCPLGLLLLLLAGQAALEARRSRWRRELAPGLHLRGIRDAGGRYCQEQDMCCRGRADECALPYLGATCYCDLFCNRTVSDCCPDFWDFCLGIPPPFPPVQGCMHAGRIYPIFGTYWENCNRCTCHEKGQWECDQEPCLVDPAMIKAINRGNYGWQAGNHSAFWGMTLDEGIRYRLGTIRPSSSVMNMNEIYTVLGQGEVLPTAFEASEKWPNLIHEPLDQGNCAGSWAFSTAAVASDRVSIHSLGHMTPILSPQNLLSCDTHHQKGCRGGRLDGAWWFLRRRGVVSDNCYPFSGREQNDEASPTPRCMMHSRAMGRGKRQATSRCPNSQVDSNDIYQVTPVYRLASDEKEIMKELMENGPVQALMEVHEDFFLYQRGIYSHTPVSQGRPEQYRRHGTHSVKITGWGEETLPDGRTIKYWTAANSWGPWWGERGHFRIVRGINECDIETFVLGVWGRVGMEDMGHH

SEQ ID NO: 27 - protein, 476 aa, Bos Taurus
MWRRPLGLLLLLLLLLAGELGVGARRGRRRRELAPALHRRGIRDAGGRYCQEQDLCCRGRADDCALPYLGVTCYCDLFCNRTVSDCCPDFWDFCLGVPPPFPPIQGCMHKGRIYPVLGTYWDNCNRCTCHEKERWECDQEPCLVDEDMIEAINHGDYGWRAGNHSAFWGMTLDEGIRYRLGTVRPSSFVANMNEIHTVLGPGEVLPRTFEASEKWPNLIHDPLDQGNCAGSWAFSTAAPLPPFPAVASDRVSIHSLGHMSPVLSPQNLLSCDTHNQQGCRGGRLDGAWWFLRRRGVVSDHCYPFSGHGRDEAVPAPPCMMHSRAMGRGKRQATARCPNSYVHANDIYQVTPAYRLGSNEKEIMKELMENGPVQALMEVHEDFFLYQSGIYSHTPVSLGRPERYRRHGTHSVKITGWGEETLPDGRTIKYWTAANSWGPAWGERGHFRIVRGANECDIESFVLGVWGRVGMEDMGHH

SEQ ID NO: 28 - protein, 464 aa, Gallus gallus
MANTALVPWVLLWLLAAVGSAARARTRRELSPGLYEHGVFDAGGSYCQRGDVCCRGRDDGCTVPYLDTICYCDLFCNRTVSDCCPDFWEYCLGIPAPFPKAPGCARSGRTYPSGATYRDNCNLCTCSPGGQWQCEDHACLMDGDLIDAVNRGNYGWRAANYSQFWGMTLEDGMRYRLGTFRPPPTVMNMNEMHMAMDSNEVLPRHFDAATKWPGMIHEPLDQGNCAGSWAFSTAAVASDRISIHSMGHMTPSLSPQNLLSCDTRNQRGCSGGRLDGAWWYLRRRGVVTDECYPFTSQDSQPAAQPCMMHSRSTGRGKRQATARCPNPQTHANDIYQSTPAYRLAPSEKEIMKELMENGPVQAILEVHEDFFLYKSGIYRHTAVAEGKGPKHQQHGTHSVKITGWGEEQLPDGQVQKYWTAANSWGRAWGEDGHFRIARGVNECEVESFVVGVWGRVSVEDMPHK

SEQ ID NO: 29 - protein, 467 aa, Macaca mulatta
MWRCPLGLLLLLSLAGHVALGAKQGRVRRELAPGLHLRGIRDAGGRYCQEQDLCCRGRADDCALPYLGAICYCDLFCNRTVSDCCPDFWDFCLGVPPPFPPIQGCMHGGRIYPVLGTYWDNCNRCTCQENRQWQCDQEPCLVDPDMIKAINQGNYGWQAGNHSAFWGMTLDEGIRYRLGTIRPSSLVMNMHEIYTVLNPGEVLPTAFEASEKWPNLIHEPLDQGNCAGSWAFSTAAVASDRVSIHSLGHMTPVLSPQNLLSCDTHQQQGCRGGRLDGAWWFLRRRGVVSDHCYPFSGRERDEAGPAPPCMMHSRAMGRGKRQATARCPNSHVNNNDIYQVTPVYRLGSNDKEIMKELMENGPVQALMEVHEDFFLYKGGIYSHTPVSLGRPERYRRHGTHSVKITGWGEETLPDGRTLKYWTAANSWGPAWGERGHFRIVRGVNECDIESFVLGVWGRVGMEDMGHH

SEQ ID NO: 30 - protein, 465 aa, Mesocricetus auratus
MWGCRLGLLLLLLAGQAALEARRSRWRRELAPGLHLRGIRDAGGRYCQEQDMCCRGRADDCALPYLGATCYCDLFCNRTVSDCCPDFWDFCLGVPPPFPPIQGCVHGGRIYPVFGTYWNNCNRCTCQKTGQWECDQEPCLVDPDMINAINRGNYGWQAGNHSAFWGMTLDEGIRYRLGTIRPSSSVMNMNEIYTVLGRGEVLPTAFEASEKWPNLIHEPLDQGNCAGSWAFSTAAVASDRVSIHSMGHMTPILSPQNLLSCDTHHQQGCRGGRLDGAWWFLRRRGVVSDSCYPFVGREQNEAGTSPRCMMHSRAMGRGKRQATSRCPNGQVDSNDIYQVTPAYRLGSDEKEIMKELMENGPVQALMEVHEDFFLYQSGIYSHTPVSQRKPEQYRRHGTHSVKITGWGEETLPDGRTIKYWTAANSWGPWWGEKGHFRIVRGTNECDIESFVLGVWGRVGMEDMGH

SEQ ID NO: 31 - protein, 467 aa, Sus scrofa
MWRCPLGLLLLLLLAGELALGARRGRGRRELAPALHLRGIRDAGGRYCQEQDLCCRGRADDCALPYLGATCYCDLFCNRTVSDCCPDFWDFCLGVPPPFPPIQGCMHRGRIYPVLGTYWDNCNRCTCQEKGQWECDQEPCLVDPDMIKAINQGNYGWRAGNHSAFWGMTLDEGIRYRLGTIRPSSSVANMNEIHTVLGPGEVLPRAFEASEKWPNLIHDPLDQGNCAGSWAFSTAAVASDRVSIHSLGHMTPVLSPQNLLSCDTHNQQGCQGGRLDGAWWFLRRRGVVSDHCYPFSGHERNEAGPAPRCMMHSRAMGRGKRQATARCPNSYVHANDIYQVTPAYRLGSNEKDIMKELMENGPVQALMEVHEDFFLYQSGIYSHTPVSHGRPERYRRHGTHSVKITGWGEETLPDGRMLKYWTAANSWGPGWGERGHFRIVRGANECDIESFVLGVWGRVGMEDMGHP

SEQ ID NO: 32 - protein, 469 aa, Ovis aries
MWRRQLGLLLLLLLLLTGELGVGARRGRRRRELAPALHQRGIRDAGGRYCQEQDLCCRGRADDCALPYLGVTCYCDLFCNRTVSDCCPDFWDFCLGVPPPFPPIQGCMHKGRVYPVLGTYWDNCNRCTCHEKEQWECDQEPCLVDEDMIKAINQGNYGWRAGNHSAFWGMTLDEGIRYRLGTVRPSSSVTNMNEIHTVLGPGEVLPRTFEASEKWPNLIHDPLDQGNCAGSWAFSTAAVASDRVSIHSLGHMSPVLSPQNLLSCDTHNQQGCHGGRLDGAWWFLRRRGVVSDHCYPFSGHGRDEAVPAPPCMMHSRAMGRGKRQATARCPNSYVHANDIYQVTPAYRLGSNEKEIMKELMENGPVQALMEVHEDFFLYQSGIYSHTPVSLGRPERYRRHGTHSVKITGWGEETLPDGRTVKYWTAANSWGPAWGERGHFRIVRGANECDIESFVLGVWGRVGMEDMGHH

SEQ ID NO: 33 - protein, 467 aa, Oryctolagus cuniculus
MWLRPRGLLLLLLLAGQVALGARRGRWRRELAPGLHLRGIRDAGGSYCQKQDLCCRGRADDCALPYLGATCYCDLFCNRTVSDCCPDFWDFCLGVPPPFPPIQGCMHGGRVYPVLGTYWDNCNRCTCQEKGRWECDQEPCLVDPDMINAINQGNYGWQAGNHSAFWGMTLEEGIRYRLGTNRPPSSVMNMNEIYTGLGSGEVLPTAFEASEKWPNLIHEPLDQGNCAGSWAFSTAAVASDRVSIHSLGHMTPVLSPQNLLSCDTHHQQGCRGGRLDGAWWFLRRRGVVSDHCYPFSGHEQDEAGPAPPCMMHSRAMGRGKRQATARCPNSHVHANDIYQVTPAYRLGSNEKEIMKELLENGPVQALMEVHEDFFLYQGGIYSHTPVSLERPERYRRHGTHSVKITGWGEETLPDGRTLKYWTAANSWGPAWGERGHFRILRGTNECDIESFVLGVWGRVGMEDMGHH

SEQ ID NO: 34 - protein, 469 aa, Capra hircus
MWRRPLGLLLLLLLLLAGELGVGARRGRRRRELAPALHQRGIRDAGGRYCQEQDLCCRGRADDCALPYLGVTCYCDLFCNRTVSDCCPDFWDFCLGVPPPFPPIQGCMHKGRIYPVLGTYWDNCNRCTCHEKEQWECDQEPCLVDEDMIKAINQGNYGWRAGNHSAFWGMTLDEGIRYRLGTVRPSSSVTNMNEIHTVLGPGEVLPRTFEASEKWPNLIHDPLDQGNCAGSWAFSTAAVASDRVSIHSLGHMSPVLSPQNLLSCDTHNQQGCRGGRLDGAWWFLRRRGVVSDHCYPFSGHGRDEAVPAPPCMMHSRAMGRGKRQATARCPNSYVHANDIYQVTPAYRLGSNEKEIMKELMENGPVQALMEVHEDFFLYQSGIYSHTPVSLGRPERYRRHGTHSVKITGWGEETLPDGRTVKYWTAANSWGPAWGERGHFRIVRGANECDIESFVLGVWGRVGMEDMGHH

(19) United States
(12) Patent Application Publication
(10) Pub. No.: US 2021/0379147 A1
(43) Pub. Date: Dec. 9, 2021

(54) METHOD AND SYSTEM FOR TREATING CANCER UTILIZING TINAGL1

(71) Applicant: The Trustees of Princeton University, Princeton, NJ (US)

(72) Inventors: Yibin Kang, Princeton, NJ (US); Minhong Shen, Princeton, NJ (US)

(73) Assignee: The Trustees of Princeton University, Princeton, NJ (US)

(21) Appl. No.: 17/285,188

(22) PCT Filed: Oct. 15, 2019
(86) PCT No.: PCT/US2019/056268

§ 371 Date: Apr. 14, 2021

Related U.S. Application Data
(60) Provisional application No. 62/746,358, filed on Oct. 16, 2018.

(51) Int. Cl. A61K 38/17; A61K 45/06; A61K 48/00; C07K 14/47; G01N 33/574; A61N 5/10

(52) U.S. Cl. CPC A61K 38/1709; A61K 45/06; A61N 5/10; C07K 14/47; G01N 33/57484; A61K 48/0058

(57) ABSTRACT

Disclosed is a method of treating cancer, involving the administration of a therapeutically effective amount of an inhibitor of the epidermal growth factor receptor (EGFR) pathway and the integrin/focal adhesion kinase (FAK) pathway to a patient in need of such treatment, where the inhibitor comprises at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein.

### CROSS REFERENCE TO RELATED APPLICATIONS

This application claims priority to U.S. Provisional Application No. 62/746,358 filed Oct. 16, 2018, which is hereby incorporated in its entirety by reference.

### STATEMENT REGARDING FEDERALLY SPONSORED RESEARCH OR DEVELOPMENT

This invention was made with government support under Grant No. W81XWH-13-1-0425 awarded by the U.S. Department of Defense, Army Medical Research & Materiel Command. The government has certain rights in the invention.

### SEQUENCE LISTING

The present application is being filed along with a Sequence Listing in electronic format. The Sequence Listing is provided as a file entitled PRIN-65276_ST25.txt, created Oct. 14, 2019, which is approximately 73,879 bytes in size. The information in the electronic format of the Sequence Listing is incorporated herein by reference in its entirety.

### FIELD OF THE INVENTION

The present invention relates to a method for treating individuals with cancers or related diseases, and specifically for treating patients with cancer by providing a therapeutic amount of agent containing at least a fragment of a Tinagl1 protein, which acts as an inhibitor of both the EGFR pathway and the integrin/FAK pathway.

### BACKGROUND

As one of the most common malignant diseases among women, breast cancer also displays high degree of diversity in terms of pathological characteristics, disease progression and response to treatments. Using increasingly sophisticated gene expression profiling techniques, breast tumors have been classified into different subtypes with distinct clinical outcomes. The most common molecular classification identifies five distinct subtypes, based on the expression levels of estrogen or progesterone receptors (ER or PR) and human epidermal growth factor receptor 2 (HER2) (Sorlie et al., 2001). Of particular clinical interest is the breast cancer subtype characterized by the absence of all three receptors—triple negative breast cancer (TNBC), a heterogeneous subtype that is observed in approximately 12-17% of all breast cancer cases (Foulkes et al., 2010; Mayer et al., 2014). TNBC is particularly concerning since these patients experience worse prognosis than any other subtype owing to two major factors: higher rates of recurrence as well as limited therapeutic options (Collignon et al., 2016). Moreover, TNBC tumors are usually more aggressive and more likely to metastasize than other subtype of breast cancer. Both innate and adaptive drug resistance is commonly observed in breast cancer patients with metastatic TNBC (Gonzalez-Angulo et al., 2007; Lehmann and Pietenpol, 2014). Therefore, effective targeted therapies for TNBC are urgently needed.

Amplification or mutations of the epidermal growth factor receptor (EGFR) gene are associated with many types of cancer (Arteaga and Engelman, 2014). EGFR signaling is often highly active in TNBC (Costa et al., 2017), and is correlated with poor prognosis in basal-like TNBC (Park et al., 2014). Although small molecule inhibitors and blocking antibodies against EGFR have been shown to significantly suppress TNBC cells growth in vitro (Bao et al., 2017), these agents showed limited effect on the clinical outcome in TNBC patients (Costa et al., 2017), possibly due to compensation by other oncogenic pathways in vivo (Rexer et al., 2009).

Previous studies have demonstrated that EGFR signaling is extensively connected to integrin signaling in regulating many cellular functions, such as cell adhesion, migration, and oncogenic transformation (Desgrosellier and Cheresh, 2010). Overexpression and activation of integrin signaling has also been associated with the malignant features of, e.g., breast cancer (Desgrosellier and Cheresh, 2010). One of the most prominent downstream effectors of integrin signaling is focal adhesion kinase (FAK), which has also been shown to drive, e.g., breast cancer progression (Sulzmaier et al., 2014) and correlate with poor clinical outcome in, e.g., breast cancer (Alexopoulou et al., 2014; Almstedt et al., 2017), particularly in TNBC patients (Golubovskaya et al., 2014). While these findings support the rationale of targeting the integrin/FAK signaling cascade in TNBC, clinical trials targeting integrin signaling again showed limited efficacy (Carter, 2010), similar to the largely negative outcome of single agent trials of EGFR inhibitors in TNBC (Costa et al., 2017).

Thus, treatments for cancers, such as TNBC, are still needed and desirable.

### BRIEF SUMMARY

Disclosed is a method for treating cancers, such as TNBC, comprising administering to a patient in need thereof a therapeutically effective amount of an inhibitor of the EGFR pathway and the integrin/FAK pathway, wherein the inhibitor comprises at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein. Optionally, the inhibitor interacts with EGFR, integrin α5β1, αvβ1, or a combination thereof. Optionally, the patient may be a mammal, such as a human female. Optionally, the patient has previously been diagnosed with a cancer such as TNBC. Optionally, the patient has previously been diagnosed with a cancer having active Integrin signaling, active EGFR signaling, or a combination thereof.

Optionally, the Tinagl1 protein is human Tinagl1 protein [SEQ ID NO.: 1]. Optionally, the inhibitor is produced by recombinant expression, such as in mammalian, insect, bacterial, or yeast cells. Optionally, the recombinant expression occurs in various bacteria or yeast cells, such as E. coli, N. lactamdurans, S. cerevisiae, and K. lactis, or mammalian cells such as Chinese hamster ovary (CHO) cells, or Human embryonic kidney (HEK) cells. Optionally, the inhibitor is produced by endogenous expression of Tinagl1 in human cells or tissues. Optionally, the inhibitor is extracted from a native protein source (e.g., mammalian cell cultures, tissues or bodily fluids such as blood), or overexpression in a model system (e.g., such as bacteria, yeast, insect or mammalian cells).

Optionally, at least one additional therapeutic agent is administered to the patient. The additional therapeutic agent may be a chemotherapeutic agent, an anti-cell proliferation agent, a gene therapy agent, and/or an immunotherapy agent. Optionally, the treatment method also includes administering ionizing radiation to the patient. Optionally, the patient is not administered any cancer therapeutic agent except the inhibitor.

Optionally, the inhibitor is administered intravenously, subcutaneously, intramuscularly, intralesionally, intraperitoneally, via liposomes, transmucosally, intestinally, topically, via nasal route, orally, via anal route, via ocular route, or via otic route.

Optionally, the method also includes administering to the patient an additional therapeutically effective amount of the inhibitor at a second point in time after the therapeutically effective amount of the inhibitor was first administered.

Optionally, the method also includes determining an expression level of a Tingal gene or of a Tingal protein or a variant thereof of the subject.

Also disclosed is an isolated recombinant protein, comprising the first 94 amino acids of a Tinagl1 protein, a fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein. Optionally, the isolated recombinant protein is a full length Tinagl1 protein, such as the human Tinagl1 protein [SEQ ID NO.: 1]. Optionally, the isolated recombinant protein may be present by itself, or combined with a pharmaceutically acceptable carrier.

Also disclosed is a therapeutic dose involving the isolated recombinant protein described above, and a pharmaceutically acceptable carrier.

Also disclosed is a method for treating a cancer in a subject via gene therapy. The method involves administering to a patient a pharmaceutical composition comprising a viral or non-viral delivery system with a gene under control of a promoter sequence, the gene capable of expressing at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein.

Also disclosed is a stable cell line that includes a gene under control of a promoter sequence, the gene capable of expressing at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein.

Also disclosed is a method of manufacturing a composition for treating cancer. The method involves first providing a cell from a stable cell line capable of overexpressing at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein. Then, the cell is grown, after which the overexpressed protein can be extracted. Optionally, the overexpression can be controlled via the introduction of, e.g., doxycycline.

Also disclosed is are two methods for ex vivo screening of cancers. In the first method, the level of expression, in the subject, of at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein is measured in a sample of bodily fluid received from a subject, and then a determination is made as to whether the measured level of expression is below a predetermined threshold.

In the second method, a measurement of a level of expression, in a subject, of at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein is received, and a determination that the subject should be treated for cancer is made when the measured level of expression is below a predetermined threshold.

### BRIEF DESCRIPTION OF DRAWINGS

FIG. 1 is an illustration of various domains in a human Tinagl1 protein.

FIG. 2 is a Kaplan-Meier plot of disease-free survival (DFS) of breast cancer patients stratified by TINAGL1 mRNA expression level in tumor samples, where TINAGL1 mRNA level was assessed by RT-qPCR and normalized by GAPDH mRNA level.

FIG. 3A is a graph illustrating primary tumor volumes measured weekly when LM2 cell lines with either vector (210) or Tinagl1 (220) stably expressed were inoculated into NSG mice by mammary fat pad injection (10⁴ tumor cells per injection, n=12 mice per group).

FIG. 3A is a scatter plot illustrating primary tumor volumes at the last time point measured when LM2 cell lines with either vector (215) or Tinagl1 (225) stably expressed were inoculated into NSG mice by mammary fat pad injection (10⁴ tumor cells per injection, n=12 mice per group).

FIG. 4A is a Kaplan-Meier plot of tumor-free survival of PyMT;Tinagl1-KO (330), HET (320), or WT (310) mice. WT=12 mice, HET=5 mice, and KO=15 mice.

FIG. 4B is a graph of total tumor burden, as measured once per week, for weeks 7-15, in PyMT;Tinagl1-KO (333), HET (323), or WT (313) mice. WT=6 mice, HET=5 mice, and KO=7 mice.

FIG. 4C is a scatter plot of metastatic lung nodules at the end point, from dissected lungs in PyMT;Tinagl1-KO (336), HET (326), or WT (316) mice. WT=6 mice, HET=5 mice, and KO=7 mice.

FIG. 5 is a graph of tumor growth rates in three groups of mice modified to express Tinagl1 unless given doxycycline, as measured once per week for 6 weeks: 1) constitutively high expression of Tinagl1 (No DC) (410); constitutively low expression of Tinagl1 (+DC) (420), and 3) low expression for 2 weeks, then high expression of Tinagl1 after tumors are well established (430).

FIG. 6 is a graph of lung metastasis burden measured by BLI every week, for NSG mice under one of three treatment regimens: 1) PBS immediately following injection (510), 2) PBS for the first two weeks followed by r-Tinagl1 treatment (520), and 3) r-Tinagl1 immediately after injection (530).

FIGS. 7A-7D are graphs indicating the percentage of Ki67⁺ (FIGS. 7A and 7B) and cleaved caspase-3⁺ (FIGS. 7C, 7D) cells counted in primary tumor (FIGS. 7A, 7C) and spontaneous lung metastasis (FIGS. 7B, 7D) samples from mice treated with r-Tinagl1(711, 721, 731, 741) or PBS (710, 720, 730, 740) collected for H&E and IHC staining with indicated antibodies.

FIGS. 8A and 8B are silver staining (8A) and WB (8B) validation following immunoprecipitation, where LM2 cells expressing the C-terminal HA tagged Tinagl1 (Tinagl1-HA) were lysed and immunoprecipitated (IP) with IgG (control) or anti-HA antibody.

FIGS. 8C and 8D are a mass spectrometry profiles of the Tinagl1 interacting partners EGFR (8C) and integrin β1 subunit (8D).

FIGS. 8E and 8F are images of WB analyses where LM2 cells expressing Tinagl1-HA stably were lysed and IP with IgG or anti-HA antibodies, then the IP samples were subjected to WB analysis with indicated antibodies to detect the interaction with EGFR (8E) and integrin β1 subunit (8F).

FIGS. 8G and 8H are images of WB analyses where HCC1937 cells were lysed with 1 ml of IP lysis buffer, then 200 μL of 100× concentrated culture media was mixed with cell lysate and samples were split into three tubes and IP with 2 μg of IgG, anti-integrin β1 subunit or EGFR antibodies respectively, then analyzed by western blot and probed with indicated antibodies.

FIGS. 9A and 9B FIGS. 8E and 8F are images of WB analyses where LM2 cells expressing Tinagl1-HA stably were lysed and IP with IgG or anti-HA antibodies, then the IP samples were subjected to WB analysis with indicated antibodies to detect the interaction with integrin α5 subunit (9A) and integrin av subunit (9B).

FIGS. 10A-10C are the GSEA results demonstrating the enrichment of the indicated gene setsin the ranked gene list of Tinagl1-expressing vs. control LM2 cells where Lung metastatic nodules formed by LM2 cells stably expressing the vector control or Tinagl1 were dissected and digested, then tumor cells were isolated and total RNA was extracted and subjected to gene expression profiling analysis, including EGF_UP (10A) (NES=−2.08, P=0, q=0), EGFR INHIBITOR_DOWN (10B) (NES=−2.36, P=0, q=0), and FAK INHIBITOR_DOWN (10C) (NES=−1.82, P=0, q=0.007).

FIG. 11A is an image of WB analyses where LM2 cells with or without 1 hour pre-treatment of the indicated amount of r-Tinagl1 were then treated with indicated amount of EGF for another 10 min, then lysed.

FIG. 11B is an image of WB analyses where vector control or Tinagl1-HA expressing LM2 cells were cultured for 48 hr, followed by 10 min treatment of 1 ng/ml EGF.

FIG. 11C is a graph quantifying the level of p-EGFR after EGF treatment.

FIG. 12 is an image of WB analyses where a 6-well plate was coated with 10 μg/ml of FN or 10 μg/ml of FN+r-Tinagl1, then SUM159-M1a cells were serum starved for 24 hr, and then seeded on the plate with the media contain 10% FBS and 1 ng/ml of EGF, and then 3 hr after seeding, the cells were treated with 50M FAK inhibitor 14 (FAKi) for 2 hr, followed by 1 hr treatment of 100 nM Erlotinib (Erlo), 1 μg/ml r-Tinagl1 alone or combined, and finally the cells were treated with 1 ng/ml of EGF for another 10 min, and then collected for WB analysis.

FIG. 13A is an image of IP and WB assays where EK293T cells expressing Tinagl1-HA were lysed and 5 μg of recombinant EGF (r-EGF) was added into the lysate, which was then subjected to the assays.

FIG. 13B is an image of a WB assay where 5 μg each of recombinant His-tagged EGFR protein and recombinant Tinagl1 protein were added into 1.5 ml of PBS. 100 μl of combined solution was transferred to a new tube and served as input, and the rest was split into two tubes and IP with 2 μg of IgG or His antibody respectively; the IP samples were washed with PBS and analyzed with WB.

FIG. 13C is an image of IP and WB assays where HEK293T cells co-expressing Tinagl1-HA and GFP-EGFR were lysed and divided into multiple groups, then PBS or indicated amount of proteins was added into each group, then IP followed by WB assays were performed.

FIG. 13D is a graph quantifying the ability of the indicated protein to compete with Tinagl1-EGFR interaction, by measuring GFP-EGFR level in the groups from FIG. 13C.

FIG. 14A is an image of WB analyses where LM2 cells were transfected with plasmids to overexpress GFP-EGFR and EGFR-Myc. 48 hr after transfection, the cells were treated with or without 1 μg/ml of r-Tinagl1 for 1 hr, followed by 10 min of 1 ng/ml EGF treatment, then collected and immunoprecipitated with either IgG or anti-Myc antibody, after which the IP samples were subjected to WB analysis.

FIG. 14B is a graph illustrating the quantified amount of EGFR-GFP that interacts with EGFR-Myc, normalized to PBS treatment group.

FIG. 14C is an image of WB analyses where LM2 cells stably expressed EGFR-Myc were pretreated with PBS or 1 μg/ml of r-Tinagl1 for 1 hr, treated with 1 ng/ml of EGF for another 10 min, then cells were collected and the dimers were cross-linked with disuccinimidyl suberate (DSS) treatment, followed by WB analysis.

FIG. 14D is a graph illustrating the ratio of dimerized EGFR in each treatment group that was quantified.

FIG. 15A is an graph of attached cells quantified by luciferase assay where a 96-well plate was first coated with 10 μg/ml of FN, then SUM159-M1a cells were incubated with 10 μg/ml of indicated antibodies or r-Tinagl1 at 4 C for 30 min, after when cells were seeded on the plate at 30 k cells per well, and 30 min after seeding, the plate was washed with PBS and quantified by luciferase assay.

FIG. 15B is an image of WB analyses where 5 μg of FN was added into the lysate of HEK293T cells expressing Tinagl1-HA, then the lysate was immunoprecipitated with indicated antibodies, followed by WB analysis.

FIG. 16A is an image of WB analyses where HEK293T cells overexpressing either wild type β1 or β1-mutant (β1-M) were lysed, then 5 μg of FN was added into each sample, followed by IP and WB analysis.

FIG. 16C is an image of WB analyses where HEK293T cells overexpressing wild type β1 and Tinagl1-HA or integrin β1-M subunit and Tinagl1-HA were lysed for IP, the analyzed with WB.

FIG. 17A is an image of WB analyses where NSG female mice were injected with 10⁴ LM2 cells by MFP injection, and intravenously treated with the indicated reagents twice per week when tumors reached to 2 mm in dimeter, then after 5 weeks of the treatments, the primary tumors of each group were collected and activation of EGFR and FAK in primary tumor of each group were tested by WB.

FIG. 17B is a graph illustrating tumor volumes of each treatment group from FIG. 17A.

FIG. 17C is a graph, quantifying ex vivo BLI results where lungs in mice from FIG. 17A were collected and spontaneous metastasis was examined at the endpoint.

FIG. 17D is an image of WB analyses where NSG female mice were injected with 10⁴ M1a cells by MFP injection, and treated with the indicated reagents twice per week when tumors reached to 2 mm in diameter, then after 5 weeks of the treatments, the primary tumors of each group were collected and activation of EGFR and FAK in primary tumor of each group was tested by WB.

FIG. 17E is a graph illustrating tumor volumes of each treatment group from FIG. 17D.

FIGS. 17F-17I are graphs quantifying positives cells per field where primary tumors were collected from indicated treatment groups from FIG. 17D and then subjected to IHC staining with indicated antibodies, including % Ki67⁺ (17F), % Casp3⁺ (17G), % CD31⁺ (17H), and % α-SMA⁺ (17I).

FIGS. 18A-18C are Kaplan-Meier plots of DFS (18A), DMFS (18B), and LMFS (18C) of TNBC patients stratified by Tinagl1 protein levels, activated EGFR and FAK.

### DETAILED DESCRIPTION

For various cancers, many extracellular matrix (ECM) proteins are ligands and regulators of integrin/FAK signaling and are involved in various aspects of cancer progression, including growth, survival, tumor invasion and metastasis.

The term “conservative substitution” as used herein refers to an amino acid replacement in a protein that changes a given amino acid to a different amino acid with similar biochemical properties (e.g., charge, hydrophobicity and size). Conservative substitutions include artificial mutations, deletions, or additions as well as natural changes, including changes from other species.

The term “epidermal growth factor receptor” (“EGFR”) as used herein refers to a gene that encodes a membrane polypeptide that binds, and is thereby activated by, epidermal growth factor (EGF). EGFR is also known in the literature as ERBB, ERBB1 and HER1. An exemplary EGFR is the human epidermal growth factor receptor. Binding of an EGF ligand activates the EGFR (e.g., resulting in activation of intracellular mitogenic signaling, autophosphorylation of EGFR). One of skill in the art will understand that other ligands, in addition to EGF, can bind to and activate the EGFR. Examples of such ligands include, but are not limited to, amphiregulin, epiregulin, TGF-α, betacellulin (BTC), and heparin-binding EGF (HB-EGF).

The term “EGFR pathway” as used herein refers to the signaling pathway downstream of EGFR that is initiated through binding to EGFR. As understood by those of skill in the art, activation of EGFR can lead to homodimerization/heterodimerization, phosphorylation of specific tyrosine residues, and recruitment of several proteins at the intracellular portion of the receptors.

The term “Focal adhesion kinase” (“FAK”) as used herein refers to a cytoplasmic tyrosine kinase identified as a mediator of intracellular signaling by integrins.

The term “fusion protein” as used herein refers to a genetically engineered protein that is encoded by a nucleotide sequence made by a joining together two or more complete or partial genes or a series of nucleic acids. Alternatively, a fusion protein may be made by joining together two or more of heterologous peptides.

The term “homology” as used herein refers to a degree of identity. There may be partial homology or complete homology. A partially identical sequence is one that is less than 100% identical to another sequence.

The term “Integrin” as used herein refers to a family of cell surface receptors involved in mediating cellular interactions with extracellular matrix (ECM) as well as cell-cell interactions. Each integrin is a heterodimeric integral protein complex composed of an alpha chain and a beta chain, both of which are transmembrane glycoproteins with a single membrane-spanning segment and generally a short cytoplasmic domain.

The term “Integrin/FAK pathway” as used herein refers to a signaling pathway wherein integrin activation of FAK can trigger a subsequent signaling cascade in one or more various cell processes, such as survival signaling, growth, angiogenesis, migration, and invasion. For example, in integrin-mediated cell adhesion, FAK is activated via disruption of an auto-inhibitory intra-molecular interaction between its amino terminal FERM domain and the central kinase domain. The activated FAK forms a complex with Src family kinases, which initiates multiple downstream signaling pathways through phosphorylation of other proteins to regulate different cellular functions. Multiple downstream signaling pathways have been identified to mediate FAK regulation of migration of various normal and cancer cells.

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other component normally present in a solution of the same. The terms “isolated” and “purified” do not encompass nucleic acids or polypeptides present in their natural source.

The term “pharmaceutically acceptable” as used herein with respect to an amount or substance means that an amount or substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “primer” refers to an oligonucleotide which is capable of acting as a point of initiation of synthesis when placed under conditions in which primer extension is initiated. An oligonucleotide “primer” may occur naturally, as in a purified restriction digest or may be produced synthetically.

The term “protein” or its interchangeably used term “polypeptide” as used herein refer to a molecule composed of monomers (amino acids) linearly linked by amide bonds (also known as peptide bonds). Post-translational modifications of the polypeptide, for example, glycosylations, acetylations, phosphorylations, and the like are also encompassed. The terms “protein” or “polypeptide” also includes variants which should encompass any polypeptide comprising, or alternatively or preferably consisting of, any natural or genetically engineered polypeptide having more than 70%, preferably more than 80%, even more preferably more than 90%, again more preferably more than 95%, and most preferably more than 97% amino acid sequence identity with the sequence of the polypeptide. Preferred methods of generating a variant of a polypeptide is by genetic engineering, preferably by insertion, substitution, deletion or a combination thereof.

The term “recombinant” as used herein with respect to a polypeptide or protein means that a polypeptide or protein is derived from recombinant (e.g., microbial or mammalian) expression systems, where “microbial” refers to recombinant polypeptides or proteins made in bacterial or fungal (e.g., yeast) expression systems.

The term “secreted” as used herein includes a protein that is transported across or through a membrane, including transport as a result of signal sequences in its amino acid sequence when it is expressed in a suitable host cell. “Secreted” proteins include without limitation proteins secreted wholly (e.g., soluble proteins) or partially (e.g., receptors) from the cell in which they are expressed. “Secreted” proteins also include without limitation proteins which are transported across the membrane of the endoplasmic reticulum.

The term “subject” as used herein refers to an animal. Preferably, the animal is a mammal. Mammals include humans and non-human mammals, such as murines, simians, lab animals, farm animals, sport animals, and pets. Non-limiting examples of a subject includes primates (e.g., humans), cows, sheep, goats, horses, dogs, cats, rabbits, rats, mice, fish, birds and the like. In a preferred embodiment, the subject is a human. The terms “subject” and “patient” are used interchangeably herein to refer to a vertebrate, preferably a mammal, and more preferably a human.

The term “Tubulointerstitial Nephritis Antigen-Like Protein 1” (“Tinagl1”) or “Tinagl1 protein” as used herein refers to an extracellular matrix protein that plays an important role in cell adhesion and therefore modulates cell proliferation, migration, and differentiation. The Tinagl1 gene (that encodes for Tinagl1) is broadly conserved; for example, the human Tinagl1 gene has orthologs in over 250 species, with homologs in chimpanzee, dog, cow, mouse, rat, chicken, zebrafish, fruit fly, mosquito, C. elegans, and frog.

The term “about” as used herein means within 20%, preferably within 10%, and more preferably within 5% of a given value or range. For example, “about 10%” means from 8% to 12%, preferably from 9% to 11%, and more preferably from 9.5% to 10.5%. When the term “about” is associated with a range of values, e.g., “about X to Y %”, the term “about” is intended to modify both the lower (X) and upper (Y) values of the recited range. For example, “about 0.1 to 10%” is equivalent to “about 0.1% to about 10%”.

Tubulointerstitial nephritis antigen like 1 (Tinagl1), a secreted extracellular protein, was initially identified as a novel putative component of the ECM. Secretion of Tinagl1 is mediated by Sec23a-dependent ER-Golgi protein trafficking pathway, and Tinagl1 knockdown in breast cancer cells led to increased metastatic lung colonization. Tinagl1 expression is correlated with good prognosis in cancers, and specifically breast cancer, particularly among TNBC patients. Moreover, Tinagl1 inhibits progression of cancers like TNBC by simultaneously blocking EGFR and integrin/FAK signaling with distinct mechanisms. Importantly, therapeutic treatment of recombinant Tinagl1 significantly suppresses cancer growth and metastasis in mouse models, supporting its potential development as a novel therapeutic agent for cancers.

Thus, embodiments of the disclosed method may involve determining an expression level of a Tingal gene or of a Tingal protein or a variant thereof of the subject, at some point during the method. In some embodiments, the determination is made prior to any treatment occurring. In some embodiments, multiple determinations are made, including before, during, and after treatments.

The disclosed method for treating cancers, including breast cancers such as TNBC, comprising administering to a patient in need thereof a therapeutically effective amount of an inhibitor of the EGFR pathway and the integrin/FAK pathway, wherein the inhibitor comprises at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein, such as 91% or above homology, 92% or above homology, 93% or above homology, 94% or above homology, 95% or above homology, 96% or above homology, 97% or above homology, 98% or above homology, or 99% or above homology.

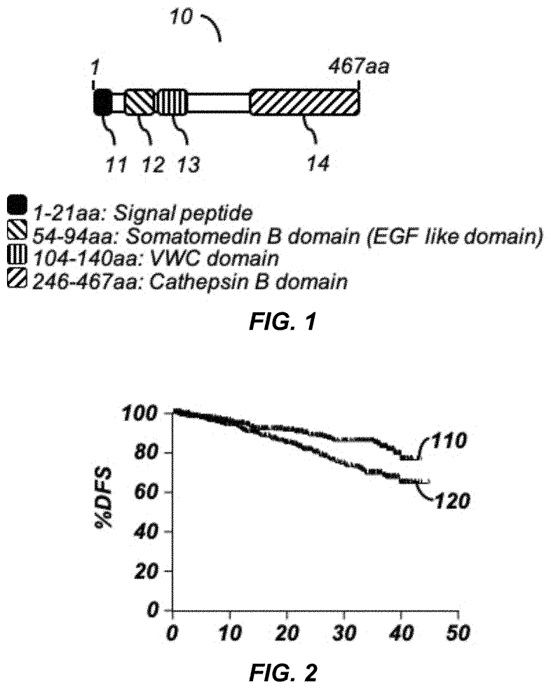

FIG. 1 is an illustration depicting various domains found in the 467 amino acids (1-467aa) of the human Tinagl1 protein (10) [SEQ ID NO.: 1]. At least four domains (11, 12, 13, 14) can be found in the Tinagl1 protein (10). A signal peptide (11) is found at 1-21aa; a Somatomedin B domain (12), which is an EGF-like domain, is found at 54-94aa; a Von Willebrand factor type C (VWC) domain (13) is found at 104-140aa; and a Cathepsin B domain (14) is found at 246-467aa.

The functional domains of the Tinagl1 protein were mapped against EGFR, Integrin β1, and Integrin α5, and the results can be seen in Table 1, below. Specifically, it can be seen that the signal peptide (11) and Somatomedin B domain (12) are required to interact with EGFR, Integrin, and Integrin.

TABLE 1

| | Interaction Ability | | |
|---|---|---|---|
| Tinagl1 Mutants | EGFR | Integrin β1 | Integrin α5 |
| Full Length | + | + | + |
| Δ1-21 | − | − | − |
| Δ22-53 | + | + | + |
| Δ54-94 | − | − | − |
| Δ1-94 | − | − | − |
| Δ104-140 | + | + | + |
| Δ246-467 | + | + | + |

The inhibitor may interact with EGFR, integrin α5β1, and/or αvβ1. In some embodiments, the inhibitor interacts with all three, while in others, it only interacts with one or two.

The inhibitor may be produced by recombinant expression, and may occur in mammalian, insect, bacterial, or yeast cells. In certain embodiments, the recombinant expression occurs in various bacteria or yeast cells, such as E. coli, N. lactamdurans, S. cerevisiae, and K. lactis, or mammalian cells such as Chinese hamster ovary (CHO) cells, or Human embryonic kidney (HEK) cells. In some embodiments, the inhibitor is produced by endogenous expression of Tinagl1 in human cells or tissues. Once the inhibitor is produced, the method may include extracting the protein from a native protein source (e.g., mammalian cell cultures, tissues or bodily fluids such as blood), or following overexpression in a model system (e.g., such as bacteria, yeast, insect or mammalian cells).

In certain embodiments, the inhibitor is an isolated recombinant protein. In some embodiments, the inhibitor includes a protein or peptide tag, which can include any appropriate protein or peptide tag known to those of skill in the art, including but not limited to FLAG-tags, HA-tags, his-tags, spot-tags, maltose binding protein tags, etc. The protein may be isolated and purified in any appropriate manner known to those of skill in the art, including but not limited to various chromatography techniques including affinity chromatography, size exclusion chromatography, and high-performance liquid chromatography (HPLC). For example, when full length recombinant human Tinagl1 with 6×His tag at the C-terminus [SEQ ID NO.: 24] was expressed in HEK293T cells, the recombinant protein was then purified from culture media using Ni²⁺-NTA purification system.

The inhibitor may comprise or consist of the first 94 amino acids of a Tinagl1 protein. The inhibitor may comprise or consist of between 94 and 466 amino acids of a Tinagl1 protein. Or, the inhibitor may comprise or consist of a full length Tinagl1 protein. It is believed that the inhibitor may comprise or consist of amino acid 54 through amino acid 94 of a Tinagl1 protein.

In certain embodiments, some or all of the amino acids not found in one of the four domains (11, 12, 13, 14) are not present. For example, in one embodiment, only the signal peptide (e.g., 1-21aa in human Tinagl1) and Somatomedin B domain (e.g., 54-94aa in human Tinagl1) are present.

The Tinagl1 protein may be a mammalian protein. In some embodiments, the Tinagl1 protein is human Tinagl1 protein [SEQ ID NO.: 1]. In other embodiments, the Tinagl1 protein may be from a different species, such as Canuslupus familiaris (dog) [SEQ ID NO.: 2], Equus caballus (horse) [SEQ ID NO.: 3], Mus musculus (mouse) [SEQ ID NO.: 4], Danio rerio (Zebrafish) [SEQ ID NO.: 25], Rattus norvegicus (Brown Rat) [SEQ ID NO.: 26], Bos Taurus (Cattle) [SEQ ID NO.: 27], Gallus gallus (Red junglefowl) [SEQ ID NO.: 28], Macaca mulatta (Rhesus Macaque) [SEQ ID NO.: 29], Mesocricetus auratus (Golden hamster) [SEQ ID NO.: 30], Sus scrofa (pig) [SEQ ID NO.: 31], Ovisaries (Sheep) [SEQ ID NO.: 32], Oryctolagus cuniculus (Rabbit) [SEQ ID NO.: 33], or Capra hircus (Goat) [SEQ ID NO.: 34].

As understood by those of skill in the art, minor variations from the above-referenced sequences are envisioned, including insertions, deletions, and substitutions. In some embodiments, three or fewer insertions, deletions, or substitutions are made to the first 94 amino acids of the human Tinagl1 protein. In some embodiments, the first 94 amino acids of the Tinagl1 protein have at least a 98% sequence homology to the first 94 amino acids of the human Tinagl1 protein. In some embodiments, the first 94 amino acids of the Tinagl1 protein have at least a 97% sequence homology to the first 94 amino acids of the human Tinagl1 protein. In some embodiments, the first 94 amino acids of the Tinagl1 protein have at least a 93% sequence homology to the first 94 amino acids of the human Tinagl1 protein. In some embodiments, the first 94 amino acids of the Tinagl1 protein have at least a 91% sequence homology to the first 94 amino acids of the human Tinagl1 protein.

The patients that will be treated using the disclosed method are typically mammal, male or female. In some embodiments, the patients are human. In some embodiments are human females. In some embodiments, the patients have recently been diagnosed with a cancer, such as TNBC.

To explore the role of Tinagl1 in breast cancer, the expression profile of TINAGL1 in 839 breast tumor samples was first analyzed. See Table 2, below.

TABLE 2
(Clinicopathologic Characteristics of
the TNBC Patient Cohort used in study)

| | | |
|---|---|---|
| Characteristics | All (n = 839) | TNBC (n = 198) |

Age (Years)

| | | |
|---|---|---|
| Median | 53 | 54 |
| Interquartile Range | 45-60 | 54-61 |
| ≤50 | 340 | 84 |
| >50 | 499 | 113 |

Menopausal status

| | | |
|---|---|---|
| Yes | 486 | 108 |
| No | 353 | 89 |

Stage

| | | |
|---|---|---|
| I | 378 | 85 |
| II | 334 | 80 |
| III | 127 | 32 |

Tumor grade

| | | |
|---|---|---|
| I-II | 405 | 56 |
| III | 406 | 133 |
| Unknown | 28 | 8 |

Ki67(%)

| | | |
|---|---|---|
| ≤20 | 366 | 49 |
| >20 | 418 | 135 |
| Unknown | 55 | 13 |

Chemotherapy

| | | |
|---|---|---|
| Taxane based | 453 | 141 |
| Non-taxane based | 279 | 45 |
| No chemotherapy | 82 | 8 |
| Unknown | 25 | 3 |

Radiotherapy

| | | |
|---|---|---|
| Yes | 286 | 71 |
| No | 499 | 120 |
| Unknown | 54 | 6 |

Follow-up time

| | | |
|---|---|---|
| Median | 20 | 13.7 |
| Interquartile Range | 8.6-33.8 | 8.6-19.0 |
| Recurrence-Free Survival | 126 | 49 |

Referring to FIG. 2, the patients were stratified into two groups based on the expression level of TINAGL1, e.g., higher TINAGL1 expression (110) and lower TINAGL1 expression (120). When all patients with different subtypes of breast cancer were considered as a whole, lower TINAGL1 expression correlates with advanced tumor stages and reduced disease-free survival (DFS) (See ref. 120 in FIG. 2). When these tumors were divided into ER/PR+, HER2+, and TNBC subtypes, high expression of TINAGL1 showed particularly strong correlation with good clinical outcome in TNBC. TINAGL1-high tumors also showed a trend of good prognosis in ER/PR+ and HER2+ subtypes. See Table 3, below, showing different stages of breast cancer patients stratified by TINAGL1 mRNA expression level in tumor samples, where TINAGL1 mRNA level was assessed by RT-qPCR and normalized by GAPDH mRNA level, P-value by chi-squared test.

TABLE 3

| | | Stage I | Stage II | Stage III | |
|---|---|---|---|---|---|
| All, n = 839 | Tinagl1-hi | 231 | 145 | 43 | p < 0.001 |
| | Tinagl1-lo | 147 | 189 | 84 | |
| ER/PR⁺, | Tinagl1-hi | 120 | 93 | 34 | p = 0.089 |
| n = 494 | Tinagl1-lo | 96 | 108 | 43 | |
| HER2⁺, | Tinagl1-hi | 37 | 27 | 10 | p = 0.66 |
| n = 148 | Tinagl1-lo | 32 | 29 | 13 | |
| TNBC, | Tinagl1-hi | 55 | 32 | 11 | p = 0.001 |
| n = 197 | Tinagl1-lo | 30 | 48 | 21 | |

For the qRT-PCR analysis, total RNA was isolated using RNAeasyMinikit (Qiagen), and reverse transcript with Superscript III kit (Invitrogen). Real-time quantitative PCR was performed using the Power SYBR green PCR master mix (Applied Biosystems). All analyses were performed using an ABI 7900HT PCR machine. mRNA expression was normalized by the expression of GAPDH. qRT-PCR primers used are listed in Table 4, below.

TABLE 4
(qRT-PCR Primers)

| Gene | Forward 5′-3′ | Reverse 5′-3′ |
|---|---|---|
| hTINAGL1 | TCTTCCTCGGTCATGAACAT | TTGCCTTGGTCAAGAGGCT |
| | GCA [SEQ ID NO.: 5] | CATG [SEQ ID NO.: 6] |
| hGAPDH | TGCACCACCAACTGCTTAGC | GGCATGGACTGTGGTCATG |
| | [SEQ ID NO.: 7] | AG [SEQ ID NO.: 8] |
| mTinagl1 | TCTTTCTCCGTGAGTTGCAG | CATGGTGCCTCCTGGAGTA |
| | T [SEQ ID NO.: 9] | GC [SEQ ID NO.: 10] |
| mGapdh | TCCCACTCTTCCACCTTCGA | GGGTCTGGGATGGAAATTG |
| | TGC [SEQ ID NO.: 11] | TGAGG [SEQ ID NO.: |
| | | 12] |

A similar result was observed when TINAGL1 was tested as a prognostic marker for distant metastasis-free survival (DMFS).

Consistent with higher risk of metastatic relapse in breast tumors with lower expression of TINAGL1, highly metastatic human breast cancer cell lines express lower TINAGL1 levels than weakly metastatic cells. In the 4T1 series of isogenic mouse mammary tumor cell lines with different metastatic abilities, Tinagl1 expression levels showed an inverse correlation to metastatic ability. Collectively, these data suggest that TINAGL1 is correlated with reduced metastatic ability in breast cancer.

Thus, one of skill in the art could use Tinagl1 expression (including expression of at least the first 94 amino acids of a Tinagl1 protein or any fragments with conservative substitution showing 90% or greater homology to the first 94 amino acids of a Tinagl1 protein), any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein as a biomarker for diagnosis and therapy. For example, in an ex vivo screening of cancers, a sample of bodily fluid could be received, the level of Tinagl1 expression could be measured, and that measured level could be compared to a predetermined threshold (e.g., to determine if the expression levels are “low” or “high”). A doctor could receive the measured level of expression and make a determination for a cancer treatment regimen based on the measured level of expression (e.g., treat the cancer in one way if the expression levels are “low” and another way if the expression levels are “high”).

In one embodiment, frozen breast cancer patient samples were collected, and total RNA was extracted followed by qRT-PCR (see Table 4) to measure Tinagl1 mRNA level. Glyceraldehyde 3-phosphate dehydrogenase (GAPDH) served as internal control, although other known housekeeping genes can be used alternatively. The ratio of Tinagl1/GAPDH was calculated and ranked. Top 50% was considered as Tinagl1-high, and the bottom 50% was considered as Tinagl1-low. Immunohistochemistry (IHC) staining was performed to determine Tinagl1 protein level. Paraffin-embedded breast cancer patient primary tumor or lung samples were sliced into 4 μm thickness. The slides were baked overnight at 60° C. Next, the tissue slides were washed with PBS after deparaffinization and hydration and then boiled in citrate buffer at 100° C. for 40 min. After treated with 3% H₂O₂ for 30 min to block endogenous peroxidase, slides were incubated at 4° C. overnight with Tinagl1 (Sigma, HPA048695) antibodies. Following washes with PBS, slides were then incubated with HRP-conjugated goat anti-rabbit antibody (Genentech) for 30 min at room temperature. Sections were stained by DAB and then counterstained with Gill hematoxylin according to manufacturer's instructions. To distinguish Tinagl1 high and low patient samples, two experienced pathologists who were blind to patient status reviewed and scored IHC staining independently, using the staining index (SI), which incorporates intensity and percentage of positive tumor cells. The strength of the staining was scored as follows: 0, no staining; 1, weak; 2, moderate; 3, strong; and the percentage of cells stained was scored as follows: 0, no staining; 1, <10%; 2, 10-50%; and 3, >50% tumor cells. If there was a disagreement between the two pathologists, a third pathologist was consulted to reach a consensus. The SI was derived by multiplying the staining score and percentage score. Samples with SI greater than 4 were considered as Tinagl1 high expression.

To directly test the putative tumor- and metastasis-suppressive functions of Tinagl1, a lentiviral vector was used to stably overexpress human Tinagl1 in LM2 cell line, a highly lung-metastatic subline of MDA-MB-231 that has a low TINAGL1 expression level.

The coding sequence of human Tinagl1 was cloned from the cDNA of MDA-MB-231 cells. Cloned sequence flanked by EcoR1 and Xho1, Spe1 and Xho1, or Mlu1 and Not1 restriction sites were inserted into pRVPTO (retrovrial), pLEX-MCS (lentiviral), or pRET2 vectors respectively. Human influenza hemagglutinin (HA) tag was fused to the C-terminal of Tinagl1 and inserted into pRVPTO plasmid. For human wild type and mutant integrin subunit β1-Δ130-240aa, plasmid requested from Addgene (item #69804) was used as template. Cloned sequences were flanked by BamH1 and EcoR1, and inserted into pRVPTO backbone. For human EGFR-Myc, plasmid requested from Addgene (item #39321) was used as template. Sequence flanked by HindIII and Xho1 was inserted into pRVPTO backbone. Myc tag was then added at C-terminal. For human EGFR-GFP (item #39321), integrin subunit β1-GFP (item #69804), integrin subunit α5-GFP (item #15238), and integrin subunit αv-CFP (item #57212). The plasmids were requested from Addgene. shRNAs targeting human TINAGL1 were purchased from Sigma (TRCN0000373693, and TRCN0000073773). All plasmids were sequenced and confirmed for accuracy. PCR primers used for cloning are listed in Table 5, below.

TABLE 5
(PCR Primers)

| Gene | Forward 5′-3′ | Reverse 5′-3′ |
|---|---|---|
| TINAGL1 | ATGTGGCGATGTCCACTGGGG | GTGATGACCCATGTCCTC |
| | [SEQ ID NO.: 13] | CATG [SEQ ID NO.: |
| | | 14] |
| Wild type | ATGAATTTACAACCAATTTTC | TCATTTTCCCTCATACTT |
| Integrin | TGG [SEQ ID NO.: 15] | CGGATTG [SEQ ID |
| subunit 1 | | NO.: 16] |
| Mutant | ATGAATTTACAACCAATTTTC | TTCAGCTCTCTTGAATTT |
| Integrin | TGG [SEQ ID NO.: 17] | TAATG [SEQ ID NO.: |
| subunit 1 | ATATCTGGAAATTTGGATTCT | 18] |
| | CCAG[SEQ ID NO.: 19] | TCATTTTCCCTCATACTT |
| | | CGGATTG [SEQ ID |
| | | NO.: 20] |
| EGFR | ATGCGACCCTCCGGGACGGC | TGCTCCAATAAATTCACT |
| | [SEQ ID NO.: 21] | GC [SEQ ID NO.: 22] |

Viral production and transduction of cell lines and PDX primary cells. Virus was produced as previously described (Tiscornia et al., 2006). Briefly, lentiviral plasmids, envelope plasmid (VSVG), and gag-pol plasmid (pCMV-dR8.91) were transfected together into HEK293T cells with PEI to produce viruses. 72 hours after transfection, culture media was collected and filtered with 0.4 μm filter. Similarly, retroviral vectors were transfected into the H29 packaging cell line and viruses were collected at 72 hours after transfection as described above. The viral media was 100× concentrated via ultracentrifugation, re-suspended with PBS, and saved for infection. Target cells, which were seeded one day before, were infected with virus together with 8 μg/ml polybrene. Positive cells were selected with puromycin 72 hours after infection. For PDX primary cells infection, PDX tumors were dissected and digested into single cell suspension. Virus and 8 μg/ml polybrene was added into the cell suspension. Spin infection was then performed in conical tubes for 2 hours at 1000 g and 4° C. After spin infection, the cells were collected and counted for further experiments.

A stable cell line is disclosed, where stable cell line includes a gene under control of a promoter sequence, the gene capable of expressing at least the first 94 amino acids of a Tinagl1 protein or any fragments with conservative substitution showing 90% or greater homology to the first 94 amino acids of a Tinagl1 protein.

Generation of stable cell line for inducible Tinagl1 overexpression. LM2 cells were transfected with pUHD15-1neo plasmid to express tTA (Tetracycline-controlled transactivator, A 37 kDa fusion protein consisting of the TetR and the VP16 activation domain (AD). Binds specifically to the TRE and activates transcription in the absence of Tc or Dox). The cells were then selected with neomycin 72 hours after transfection. Single colonies were picked after selection. Reporter plasmid pRET2-luc, which expresses firefly luciferase, was transfected to verify that the cells expressed tTA. The cells were then labelled with firefly luciferase to generate LM2-tTA. pRET2-Tinagl1 plasmid was transfected into LM2-tTA cells. Single colonies were picked after puromycin selection and the inhibition of Tinagl1 expression upon doxycycline treatment was validated by western blot analysis.

Thus, one of skill in the art will recognize that a method of manufacturing a composition for treating cancer is disclosed, where the method includes (1) providing a cell from a stable cell line capable of overexpressing at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein; (2) growing the cell; and then (3) extracting the overexpressed at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein. As understood by those of skill in the art, the system can be configured to utilize various inducers to control the overexpression of the cells, including, e.g., doxycycline, various wavelengths of light (using, e.g., CRY2, LOV, DRONPA or PHYB), etc.

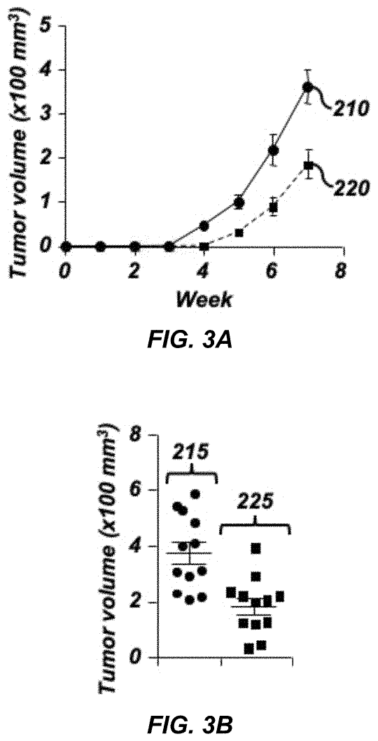

Western blotting analysis confirmed increased Tinagl1 protein expression to a level that is comparable with the endogenous level in weakly metastatic cells. Mammary fat pad (MFP) injection of the vector control and Tinagl1-overexpressing (OE) cells was performed to generate primary mammary gland tumors. These cell lines were also stably labeled with a luciferase reporter to facilitate quantification of lung metastasis by bioluminescence imaging (BLI). Referring to FIGS. 3A and 3B, Tinagl1-OE primary tumors (220, 225) grew significantly slower than the vector control cells (210, 215), which was further confirmed by tumor mass measurements at the end point of the study. FIG. 3A illustrates the tumor volume for the vector control cells (210) and Tinagl1-OE cells (220) over a period of 7 weeks, while FIG. 3B is a scatter plot of the data gathered at week 7, again indicating the tumor volume for the vector control cells (215) and Tinagl1-OE cells (225). In both figures, the differences between the two cell types are statistically significant.

Moreover, ex vivo BLI data demonstrated that Tinagl1 OE significantly inhibited spontaneous lung metastasis. To confirm these observations in additional TNBC models, Tinagl1 was overexpressed in the M1a lung-metastatic subline of the SUM159 breast cancer cell line, and the same experiments were repeated. Consistent with data from the LM2 cell line, Tinagl1 also inhibited both primary tumor growth and spontaneous lung metastasis of SUM159-M1a.

The effects of Tinagl1 OE on metastatic colonization were then specifically assessed by tail vein injection of tumor cells. Expression of Tinagl1 significantly inhibited experimental lung metastasis compared to controls, and reduced lung seeding as early as 2 hours after injection based on bioluminescent imaging (BLI), suggesting Tinagl1 affects the earliest steps of metastatic colonization.

To further confirm that Tinagl1 inhibits TNBC progression, a TNBC patient-derived xenograft (PDX, HCL001) was tested after lentiviral OE of Tinagl1. Similar to the observations above, Tinagl1 also significantly suppressed the growth of HCL001 PDX tumors in the MFP. To test Tinagl1's function in immunocompetent context, mouse Tinagl1 was overexpressed in a mouse mammary tumor cell line derived from MMTV-PyMT tumors. Again, Tinagl1 overexpression significantly suppressed primary tumor growth and lung metastasis.

Tinagl1 protein level in conditioned media of control and Tinagl-OE PyMT cells was determined by ELISA assay. 5×10⁶ cells were seeded in each 10 cm dish with 10 ml of culture media. The media was replaced with same amount of serum free media 24 hr after seeding. After another 72 hr culture, all the media was collected and concentrated to 200 μl with Amicon Ultra-15 Centrifugal Filter Units (EMD Millipore, UFC903024). Protein concentration was determined by BCA protein assay kit (ThermoFisher #23225). 50 μg of total protein was then loaded for ELISA assay (MyBioSource, MBS9331497) to measure the Tinagl1 protein amount.

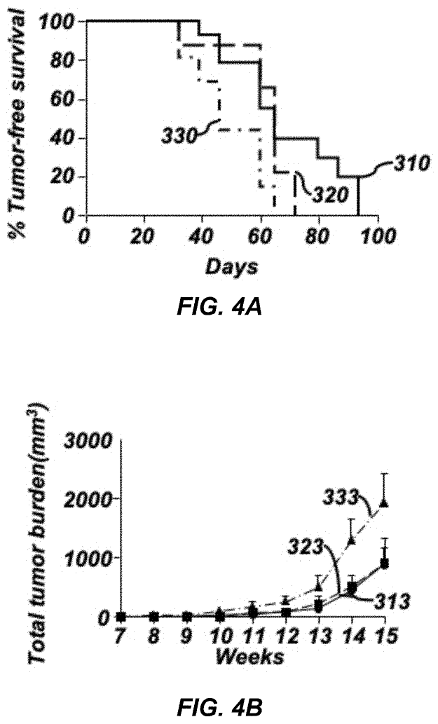

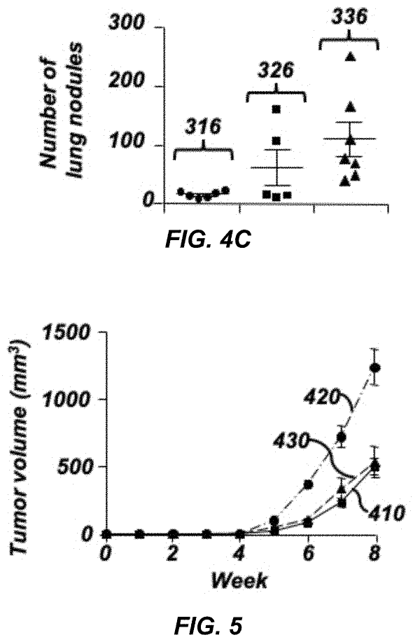

As a complementary approach to these Tinagl1 overexpression studies, it was asked if genetic knockout (KO) and/or knockdown (KD) of endogenous Tinagl1 can promote breast cancer progression. First, Tinagl1 was knocked down in MDA-MB-231 cells which have a relative higher expression level of endogenous Tinagl1, and much weaker metastatic ability compared with the lung-metastatic variant LM2. Tinagl1 KD significantly promoted lung metastasis, an observation that was consistent with a previous finding showing that Tinagl1 KD in mouse cell line 4TO7 promotes lung metastasis. Moreover, in order to investigate the role of Tinagl1 in autochthonous mammary tumors in immunocompetent animals, Tinagl1 knockout mice were crossed to MMTV-PyMT mice to generate PyMT;Tinagl1-KO animals. Tinagl1 knockout significantly promoted mammary tumor progression as well as spontaneous lung metastasis (See FIGS. 4A-4C). FIG. 4A is a Kaplan-Meier plot of tumor-free survival of PyMT;Tinagl1-KO (330), HET (320), or WT (310) mice. WT=12 mice, HET=5 mice, and KO=15 mice. In FIG. 4A, the differences between the HET (320) and KO (330) mice were statistically significant (using the Mantel-Cox test) at p<0.05, while the difference between the WT (310) and KO (330) mice were statistically significant at p<0.005. FIG. 4B is a graph of total tumor burden, as measured once per week, for weeks 7-15, in PyMT;Tinagl1-KO (333), HET (323), or WT (313) mice. WT=6 mice, HET=5 mice, and KO=7 mice. In FIG. 4B, using a student p-test, the differences between the WT (313) and KO (333) mice was statistically significant at p<0.005. FIG. 4C is a scatter plot of metastatic lung nodules at the end point, from dissected lungs in PyMT;Tinagl1-KO (336), HET (326), or WT (316) mice. WT=6 mice, HET=5 mice, and KO=7 mice. In FIG. 4B, using a student p-test, the differences between the WT (316) and KO (336) mice was statistically significant at p<0.05.

Taken together, these Tinagl1 loss-of-function studies further validate the tumor- and metastasis-suppressive role of Tinagl1.

Tinagl1 treatment can reduce tumor growth and metastasis. To assess the therapeutic potential of Tinagl1 at different stages of breast cancer, TNBC cell lines were engineered with inducible expression of Tinagl1 using a Tet-off system. In these cells, Tinagl1 expression is controlled under a Tet-responsive promoter and is turned off by doxycycline (DC) treatment, and re-activated by DC withdrawal, as validated by western blotting analysis. DC-induced expression of Tinagl1 is comparable to the level of endogenous Tinagl1 in the weakly metastatic cell line HCC1937. Cell lines with/without DC pre-treatment were orthotopically implanted into MFP to generate primary mammary tumors. The mice injected with DC pre-treated cells were then supplemented with 2 mg/ml of DC in drinking water to suppress Tinagl1 expression. Half of these mice were released from DC treatment 2 weeks following implantation once tumors had become established, while the remaining mice were maintained with DC-supplemented water. Thus, there were three groups of animals mimicking three different conditions: 1) constitutively high expression of Tinagl1 (No DC); 2) constitutively low expression of Tinagl1 (+DC), and 3) high expression of Tinagl1 after tumors are well established (Release).

Following tumor implantation, primary tumor sizes were measured on a weekly basis and mice were euthanized at week 8 to assess lung metastasis burden. As seen in FIG. 5, both constitutive (No DC) (410) and late expression (Release) (430) of Tinagl1 led to significantly reduction of primary tumor growth rate and dramatic inhibition of lung metastasis as compared to the low-Tinagl1 group (+DC) (420). Statistically, continuously high expression of Tinagl1 (No DC group) (410) showed a trend with smaller tumor than the late Tinagl1 expression (Release) (430) group at earlier time points (weeks 2 to 5). However, this difference disappeared at later time points (week 6-8), indicating the effectiveness of the tumor-inhibitory function of Tinagl1 even after tumors have been well established.

A similar treatment protocol was employed in the experimental lung metastasis colonization model using tail vein injection of the same inducible cell line. Lung metastasis was dramatically inhibited by either continuous or, to a slightly less extent, late overexpression of Tinagl1, 2 weeks after tail vein injection. To confirm the fidelity of Tinagl1 expression control by the Tet-off system in vivo, Tinagl1 expression level was evaluated in cultured cells right before injection or in lung metastasis samples. In all cases, Tinagl1 expression level faithfully reflected the DC treatment status of the cells or tissue samples.

To test if recombinant Tinagl1 protein (r-Tiangl1) treatment would slow tumor progression, full length recombinant human Tinagl1 with 6×His tag at the C-terminus (r-Tinagl1) was expressed in HEK293T cells and r-Tinagl1 was purified from the culture media using Ni²⁺-NTA purification system. r-Tinagl1 was used to treat orthotopically implanted mammary tumors. Continuous treatment of r-Tinagl1 for 7 weeks significantly inhibited primary tumor growth and spontaneous lung metastasis, while having no significant hematologic, GI tract, and liver toxicity based on body weight measurement, complete blood count (CBC), H&E and Alcian blue staining, Alanine Aminotransferase (ALT) and Aspartate Aminotransferase (AST) activity measurements.

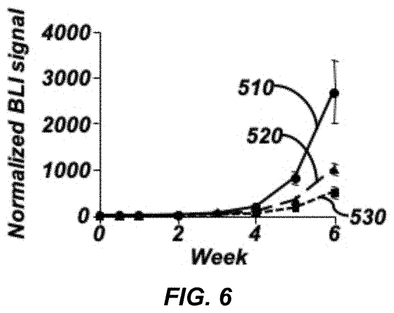

Referring to FIG. 6, to further evaluate the therapeutic potential of r-Tinagl1 treatment during metastasis, LM2 cells were injected intravenously to generate lung metastasis, and the mice were separated into three distinct treatment groups: 1) PBS immediately following injection (510), 2) PBS for the first two weeks followed by r-Tinagl1 treatment (520), and 3) r-Tinagl1 immediately after injection (530). BLI monitoring indicated that continuous r-Tinagl1 treatment significantly reduced metastatic growth (FIG. 6, 530). Beginning the r-Tinagl1 treatment two weeks into metastatic growth also dramatically decreased metastatic growth, suggesting that r-Tinagl1 has robust efficacy in inhibiting the outgrowth of established metastatic diseases (FIG. 6, 520). To confirm the BLI data, lungs were collected and fixed at the end of experiment, and both r-Tinagl1 treatment groups showed significantly reduced number of lung metastasis nodules.

To directly test the tumor-specific inhibitory effects of Tinagl1, a 3D in vitro tumor sphere growth assay was performed. Tinagl1-overexpressing SUM159-M1a tumor cells formed significant fewer and smaller spheres. Similarly, r-Tiangl1 treatment reduced the number and size of tumor spheres. These results suggest that Tinagl1 has direct inhibitory effect on tumor cells.

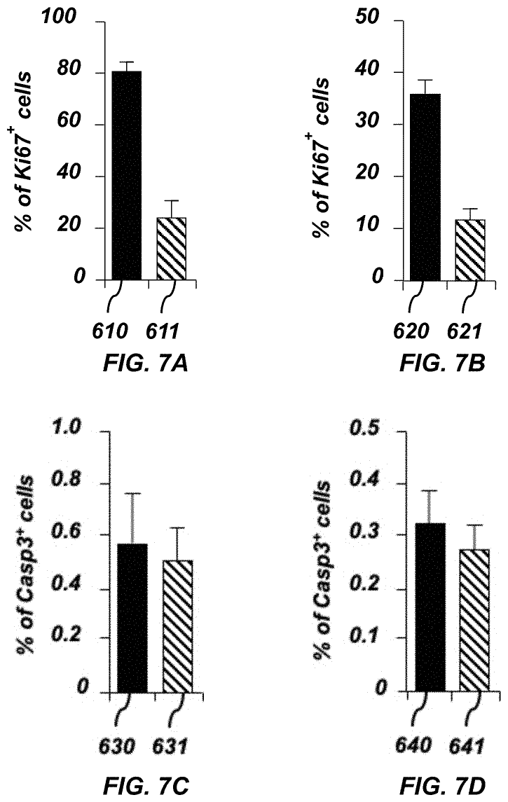

To address how Tinagl1 inhibits primary tumor growth and metastasis, primary tumors and corresponding lung samples were stained for proliferation and apoptosis markers. Referring to FIGS. 7A-7B, results indicated that primary tumors (FIG. 7A) and lung lesions (FIG. 7B) from r-Tinagl1 treated mice (611, 621) had significantly fewer Ki67⁺ cells than mice treated with PBS (610, 620). On the other hand, referring to FIGS. 7C (primary tumors) and 7D (lung lesions), there was no difference in the number of cleaved caspase-3⁺ apoptotic cells in those mice treated with r-Tinagl1 (731, 741) and those treated with PBS (730, 740), indicating that tumor cell proliferation but not survival was affected by r-Tinagl1. Similar patterns were observed in primary tumors with Tinagl1 overexpressed, and in lung sections of mice intravenously injected with Tinagl1-OE cells. Consistent results were also observed in the staining of the tissues collected from the mice injected with the SUM159-M1a cells. Taken together, the results suggested that Tinagl1 reduces tumor cell proliferation in both primary tumors and lung metastases.

Tinagl1 interacts with EGFR, integrin α5β1, and αvβ1. The underlying mechanism of Tinagl1-induced suppression of tumor growth and metastasis was investigated. Immunoprecipitation (IP) followed by mass spectrometry assay (IP-Mass) was employed to identify potential Tinagl1 interaction partners.

Immunoprecipitation (IP) and western blotting (WB) analysis. For an IP experiment, cells were collected in 1.5 ml EP tube when they reached to 100% confluency in 10 or 15 cm dish as indicated. 1 ml of IP lysis buffer (20 mM Tris pH7.4, 0.15 M NaCl, 1 mM EDTA, 1 mM EGTA, 1% Tx-100) with complete protease inhibitor cocktail (Roche, 14493900) was added and put on ice for 20 min. Meanwhile, culture media was 100× concentrated and mixed with the cell lysates. The samples were centrifuged, 100 μl of the supernatant was transferred to a new tube as input, and the rest was incubated with 2 μg of IgG, anti-HA, anti-Myc, anti-Integrin subunit β1, anti-Integrin subunit α5 or anti-EGFR (as indicated in each experiment) overnight at 4° C. (recombinant proteins may be added at this step as indicated in each experiment). 30 μL of protein A/G agarose beads (Santa Cruz, sc-2003) was then added in each sample for another 2 hours at 4° C. the next day. The beads were then washed for 5 times with IP lysis buffer. After final spin, the beads were boiled with 50 μl of 1×SDS Laemmli buffer for 5 min, and the samples were subjected to western blotting (WB). For IP between recombinant Tinagl1 and EGFR proteins, EGFR extracellular domain recombinant protein was purchased from Novoprotein (CI61). 5 μg each of recombinant His-tagged EGFR protein and recombinant Tinagl1 protein were added into 1.5 ml of PBS. 100 μl of combined solution was transferred to a new tube and served as input. The rest was split into two tubes and IP with 2 μg of IgG or His antibody respectively. The IP samples were washed with PBS and analyzed with WB.

For WB analysis, cells were collected and lysed with IP lysis buffer described above. After boiled with SDS laemmli buffer, the samples were resolved with SDS-PAGE gel and immunoblotted with standard protocols. Antibodies for IP and WB are listed in Table 6, below.

TABLE 6
(Antibodies Used)

| Antibody | Catalog# | Application |
|---|---|---|
| Tinagl1 | ProteinTech, 12077-1-AP | WB, 1:1000 |
| -actin | Abcam, ab6276 | WB, 1:10,000 |
| EGFR | Cell signaling, 4267 | WB, 1:1000/IP, |
| | | 2 g per IP |
| p-EGFR (Tyr1068) | Cell signaling, 3777 | WB, 1:1000 |
| FAK | Cell signaling, 3285 | WB, 1:1000 |
| p-FAK (Tyr397) | Cell signaling, 8556 | WB, 1:1000 |
| p-FAK (Tyr925) | Cell signaling, 3284 | WB, 1:1000 |
| AKT | Cell signaling, 4691 | WB, 1:1000 |
| p-ATK (S473) | Cell signaling, 4060 | WB, 1:1000 |
| ERK1/2 | Cell signaling, 4695 | WB, 1:1000 |
| p-ERK1/2 (Thr202/Tyr204) | Cell signaling, 4370 | WB, 1:1000 |
| Integrin 1 subunit | Cell signaling, 34971 | WB, 1:1000/IP, |
| | | 2 μg per IP |
| Integrin 5 subunit | Cell signaling, 4705 | WB, 1:1000/IP, |
| | | 2 μg per IP |
| Integrin v subunit | Abcam, ab179475 | WB, 1:1000/IP, |
| | | 2 μg per IP |
| Integrin 3 subunit | Abcam, ab190731 | WB, 1:1000 |
| Integrin4 subunit | Cell signaling, 8440P | WB, 1:1000 |
| Integrin M subunit | Abcam, ab8878 | WB, 1:1000 |
| HA | Santa Cruz, sc-7392 | IP, 2 μg per IP |
| HA | Roche, 11867423001 | WB, 1:1000 |
| MYC | Santa Cruz, sc-40 | WB, 1:1000/IP, |
| | | 2 μg per IP |
| FLAG | Sigma, F7425 | WB, 1:1000/IP, |
| | | 2 μg per IP |
| His | Sigma, H1029 | WB, 1:5000 |
| GFP | Santa Cruz, sc-9996 | WB, 1:5000 |
| Fibronectin | ProteinTech, 15613-1-AP | WB, 1:1000 |
| EGF | Santa Cruz, sc-275 | WB, 1:1000 |
| Moue IgG | Santa Cruz, sc-2025 | IP, 2 g per IP |
| Rabbit IgG | Cell signaling, 2729 | IP, 2 g per IP |

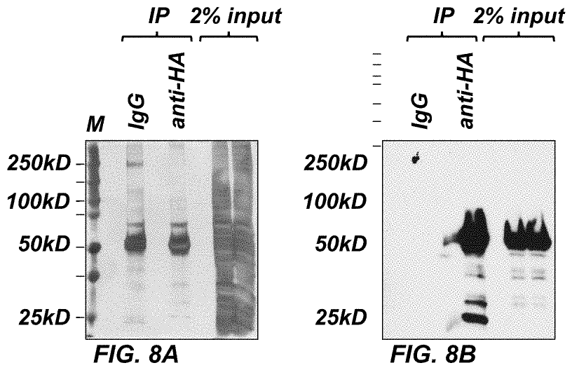

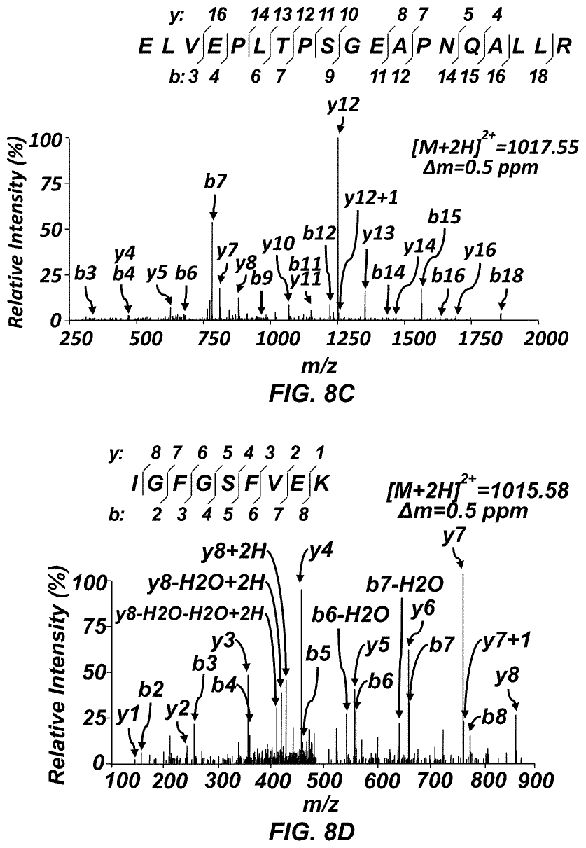

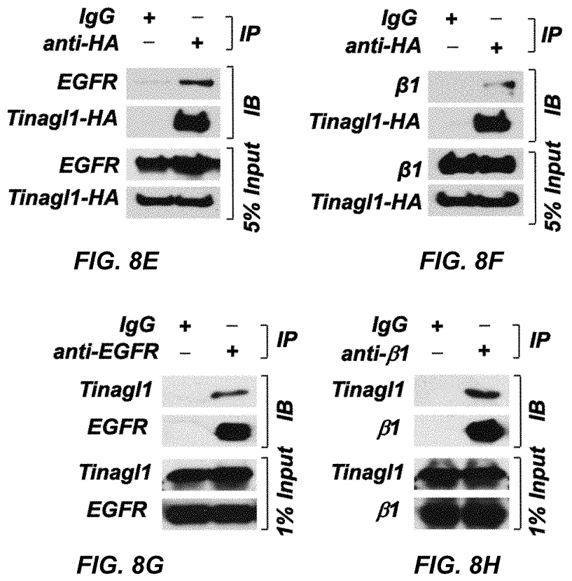

Successful IP was validated by silver staining (see FIG. 8A) and western blotting (see FIG. 8B). The immunoprecipitated samples were then subjected to mass spectrometry analysis (MS) to generate a list of Tinagl1-interacting partners. Protein network analysis of the MS result identified three pathways (Focal adhesion, ECM-receptor interaction, and PI3K-Akt pathway) significantly enriched in the candidate interacting partners, suggesting the involvement of Tinagl1 in these pathways. Of note, significant overlap of candidates was observed between the pathways, with 12 candidates present in all the pathways (EGFR, ITGB1, FN1, LAMB2, LAMB3, LAMC1, LAMC2, ITGB4, ITGA2, ITGA3, ITGA6, and THBS1). Normalized intensity ratios yielded from MS spectral counts revealed that integrin 31 subunit and EGFR were among the abundant proteins in the Tinagl1 immunoprecipitated samples (see FIGS. 8C (EGFR) and 8D (integrin β1 subunit)).

The MS results were validated using confirmatory IP experiments. LM2 cells with Tiangl1-HA stably expressed were lysed and a co-IP experiment confirmed the interaction between Tinagl1 and EGFR (see FIG. 8E) or β1 subunit (see FIG. 8F). The endogenous interaction was further conformed using HCC1937 cells that have high endogenous expression of Tinagl1. FIG. 8G shows the interaction with EGFR, while FIG. 8H shows the interaction with the β1 subunit. Taken together, these results validated the interaction detected between Tinagl1 and EGFR or integrin β1 subunit.

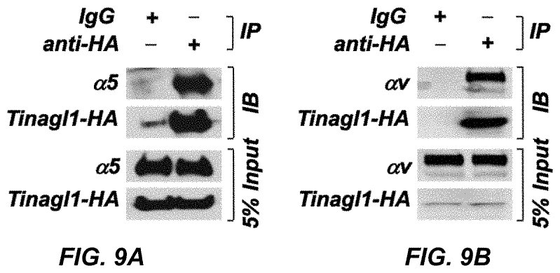

As a functional complex, integrins are composed of a and R subunits. To identify the a subunits that form heterodimer with β1 to mediate the Tinagl1 interaction, a set of a subunits with high expression abundance in breast cancer cells were tested next. Referring to FIGS. 9A-9B, the results showed that subunits α5 (FIG. 9A), av (FIG. 9B), but not the others, strongly interacted with Tinagl1. Together with the evidence that Tinagl1 interacts with β1 subunit, Tinagl1 may serve as a binding partner for both integrins α5β1 and αvβ1.

Tinagl1 inhibits EGFR and integrin/FAK signaling pathways. Microarray gene expression profiling was performed on lung metastatic lesions produced by Tinagl1-expressing or control LM2 tumor cells. Next, gene set enrichment analysis (GSEA) of C2 (curated gene sets) and C5 (GO gene sets) collections of gene sets was performed.

Microarray analysis and gene set enrichment analysis (GSEA). 2×10³ of GFP labelled LM2 cells with or without stably expression of Tinagl1 were injected into female NSG mice via tail vein. The mice were euthanized 7 weeks after injection. Lung metastatic lesions were dissected, digested, and resuspended as single cells in PBS. GFP positive tumor cells, were sorted and total RNA was isolated from these cells using the RNAeasyMinikit (Qiagen) according to manufacturer's instructions. Next, gene expression profiles were analyzed using the Agilent human GE 8×60 k two-color microarray system (Agilent G4858A-039494). The RNA samples and a universal human reference RNA (Agilent) were labeled with CTP-cy5 and CTP-cy3 using the Agilent Quick Amp Labeling Kit. Labeled samples were mixed equally and hybridized to the array. The array was then scanned with the G2505C scanner (Agilent). Data was deconvoluted and analyzed with the Genespring 13 software (Agilent). Array controls, flagged values, and expression values falling below the median value were removed. Multiple values for any given gene were collapsed into the single highest expression value. Data was extracted as a Log 2-transformed ratio of Cy5/Cy3 and was analyzed with GeneSpring software.

For GSEA, normalized microarray Log 2 ratio expression data was first rank-ordered by differential expression. Data was analyzed using GSEAv2.0. Interrogated signatures from the MySigDB v6.0 C2 curated gene sets database included EGF_UP signature, EGFR_INHIBTIRTOR DOWN signature, and the manually compiled set of FAK INHIBITOR_DOWN signature.

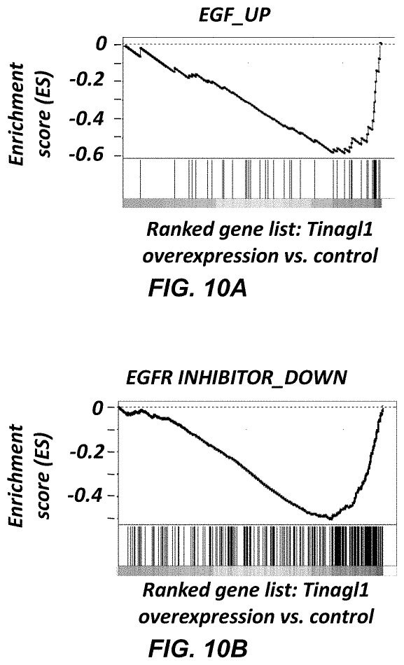

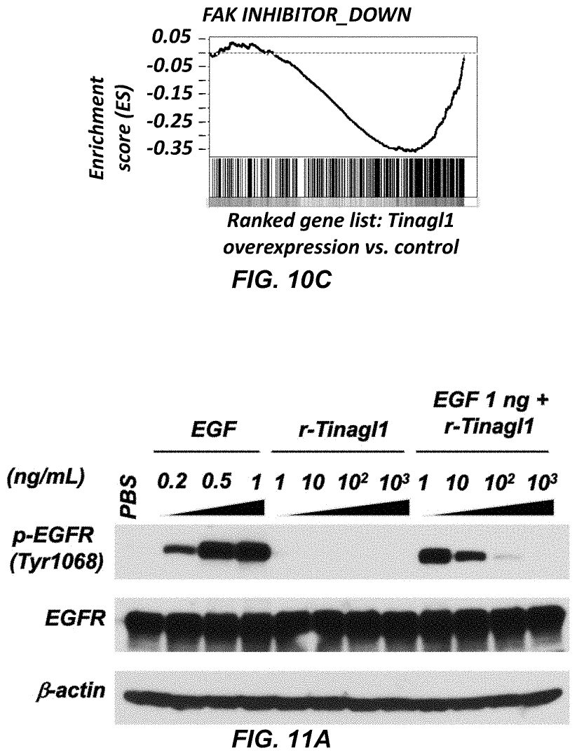

Referring to FIGS. 10A-10C, the result indicated that genes induced by EGF (FIG. 10A) or suppressed by either EGFR inhibition (FIG. 10B) or FAK inhibition (FIG. 10C) were significantly enriched in control cells compared to Tinagl1-expressing cells. To further confirm the result, a set of genes that regulated by EGFR and integrin/FAK signaling were generated.

For the EGFR or Integrin/FAK regulated gene set, it was generated with the following approaches: 1) For EGFR regulated genes, all EGFR related signatures from MySigDB v6.0 C2 curated gene sets were extracted. Gens upregulated by EGF treatment or downregulated by EGFR inhibits treatments from all the signatures were combined and termed as EGFR upregulated genes. Similarly, genes downregulated by EGF treatment or upregulated after EGFR inhibitors treatments were combined and termed as EGFR downregulated genes. 2) For the Integrin/FAK regulated genes. Microarray data from two previous studies, GSE43452 and GSE32560 were extracted and analyzed (Huang et al., 2014; Orecchia et al., 2014). Genes that were upregulated more than 4-fold by Fibronectin (FN) treatment were termed as Integrin upregulated genes. Meanwhile, genes were suppressed more than 2-fold by FAK inhibitors were defined as FAK upregulated genes and were clustered as FAK inhibitor-down signature (termed as FAK INHIBITOR_DOWN). The two lists of genes were combined and termed as Integrin/FAK upregulated genes. On the other hand, genes were downregulated more than 2-fold by FN treatment were defined as Integrin downregulated genes, and genes were upregulated more than 2-fold after FAK inhibitors treatment were defined as FAK downregulated genes. Again, these genes were combined and defined as Integrin/FAK downregulated genes. Taken together, EGFR or Integrin/FAK regulated genes from 1) and 2) respectively were combined and resulted in a list termed as EGFR or Integrin/FAK regulated genes. A heatmap was generated with GeneSpring software based on the expression of the list of genes in vector versus Tinagl1 overexpressed tumor cells.

Genes compensated by Integrin/FAK or EGFR signaling was defined as following: 1) EGFR regulated genes were defined as described above. First, the genes upregulated by EGF treatment but cannot suppressed by EGFR inhibitors treatments were clustered as EGFR inhibition resistant genes. Among them, the genes upregulated by Integrin/FAK were then picked. Top 50% of the genes were selected and termed as genes compensated by Integrin/FAK. 2) Genes upregulated by Integrin/FAK signaling but cannot suppressed by FAK inhibitors were defined as Integrin/FAK resistant genes.

Similarly, among the resistant genes, the ones that upregulated by EGFR were clustered, and the top 50% were defined as genes compensated by EGFR signaling. Heatmaps were generated based on the expression of 1) and 2) in vector versus Tinagl1 overexpressing tumor cells.

It was found that genes down regulated by EGFR and integrin/FAK signaling were significantly increased in the Tinagl1-expressing group, while genes up-regulated by either signaling programs were enriched in control group. Collectively, the results indicate that Tinagl1 was negatively correlated with EGFR and FAK activation and may inhibit both pathways.

Based on previous EGFR related signatures and the microarray data from Fibronectin (FN)- or FAK inhibitor-treated cells, two sets of EGFR and integrin/FAK crosstalk genes were identified (see Supplementary Methods): 1) Genes induced by EGF that are resistant to EGFR inhibitor treatment and up-regulated by integrin/FAK signaling (termed as genes compensated by integrin/FAK signaling); 2) Genes induced by FN that are resistant to FAK inhibitor treatment and up-regulated by EGFR signaling (termed as genes compensated by EGFR signaling).

Genes compensated by Integrin/FAK include: ACTN1, AKAP12, ARHGDIA, BCL2L1, EHD4, EPN2, F2RL1, GMDS, HMGA1, ITGA2, ITGA5, NDRG1, NFIB, PCBD1, PDLIM7, PHTF1, PPDPF, RAD23B, ROCK2, RPS6KA4, RRBP1, SMTN, TGM2, TMTC1, TPM4, and VEGFC. Genes compensated by EGFR include: ABHD2, ABHD4, AEN, AKR1B10, ALDH1A3, ALDH6A1, AP1S1, APOO, AREG, BIN1, C8orf4, C9orf114, CAMSAP1, CCDC94, CCND1, CDC27, CDC42EP2, CDK17, CDKN2AIP, CDV3, CEBPD, CHST3, COL4A1, COL4A2, CREM, CX3CL1, CXCL2, CYB561, CYP1B1, CYTH1, DCLK1, DGAT1, DHPS, DIAPHI, DLX2, DNMBP, DUSP4, EDN1, EGFR, EHBP1L1, EHD1, EPHA2, EREG, FARSA, FGF2, FOSL2, FST, FXR2, FXYD3, GLIPR1, GPR161, GPRC5A, GPX3, H3F3A, HBEGF, HCFC1R1, HES1, HIST1H2BD, HIST1H2BK, HOMER3, HSPH1, IDIl, IER3, IFIT3, IL11, IL1B, IL27RA, IL6, IL7R, IRF7, JUN, KCNJ12, LAT2, LETM1, LIF, LPCAT4, LRRFIP1, LSM4, LXN, MAFF, MAGED2, MAP2K3, MAPK1, MBD1, MBNL2, MBP, MCL1, MED20, MMP14, MT1F, MTAP, MVD, NAA15, NAV3, NCKAP1, NCLN, NFKB2, NR4A2, OGFR, OSMR, PBXIP1, PCDH7, PLAUR, PMAIP1, POR, PPP2R4, PRDX2, PTGS1, PTHLH, PTPRF, PVR, RANBP3, RANGAPI, RBMS1, RELA, RHOD, RHOF, RNF126, RPS10, RRP12, SCG5, SEC23A, SERBP1, SERPINEl, SFN, SH3BGRL3, SLC19A1, SLC25A37, SLC39A7, SNAIl, SORBS3, SRPR, SYNE2, TBC1D9, TIMM44, TNFRSF10B, TNFRSF12A, TNS4, TOMM22, TOP1, TPP1, TRAF4, TRIO, TUBB3, TUBGCP2, TUFT1, TXNRD1, UGCG, UNC93B1, USF2, VASP, ZEB1, and ZFP36L1.

Generation of these two datasets revealed that compensatory mechanisms may exist between integrin/FAK and EGFR signaling, and therefore inhibition of either pathway alone may be insufficient in a clinical setting. Interestingly, both sets of compensatory gene networks were suppressed in Tinagl1-expressing cells (FIG. 5C), further suggesting that Tinagl1 may inhibit EGFR and integrin/FAK signal pathways simultaneously.

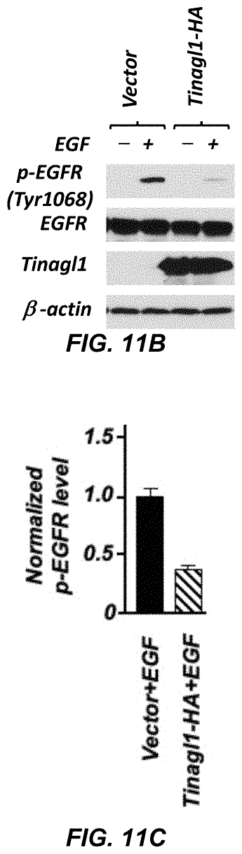

Cells were treated with r-Tinagl1 and the activity of EGFR and integrin/FAK signaling pathways was evaluated. To test EGFR activation, the LM2 cells were subjected to EGF, r-Tinagl1, or EGF/r-Tinagl1 combined treatment. Referring to FIG. 11A, EGF treatment significantly induced EGFR activation, but this induction was dramatically attenuated by r-Tinagl1 treatment in a dose-dependent manner. Referring to FIGS. 11B and 11C, EGF-dependent activation of EGFR was similarly attenuated in cells expressing Tinagl1 compared to control cells. These results were further confirmed using the SUM159-M1a cell line.

FN is the major ligand for integrin α5β1 and αvβ1, and triggers integrin/FAK signal pathway after binding. FN was employed to evaluate the effects of Tinagl1 on the integrin/FAK pathway. The result indicated that r-Tinagl1 significantly reduced FN-dependent activation of FAK signaling, as indicated by FAK phosphorylation at Tyr397, in a dose dependent manner (tested at 10 ng/mL, 100 ng/mL, 1000 ng/mL, and 10,000 ng/mL). Likewise, the activation of FAK by FN was attenuated in the cells overexpressing Tinagl1. Similar findings were observed with SUM159-M1a cells. Interestingly, EGF treatment induced FAK phosphorylation at a different location (Tyr925) without affecting the phosphorylation status of FAK Tyr397, and Tinagl1 also blocks this specific effect of EGF on FAK activation. Overall, these results revealed that Tinagl1 simultaneously inhibits EGFR and integrin/FAK signaling pathways through specific downstream mechanisms.

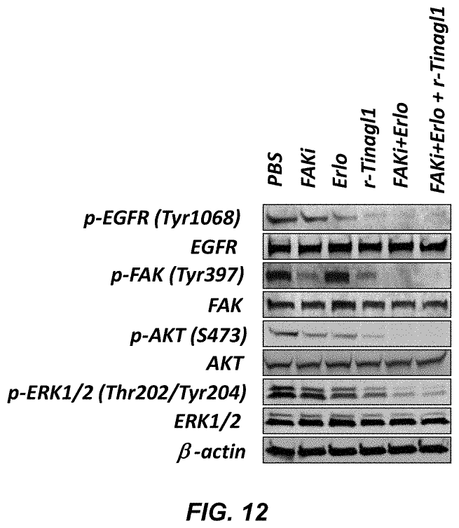

Referring to FIG. 12, r-Tinagl1 treatment inhibited the activation of both EGFR and FAK pathways, as indicated by the reduction of p-EGFR, p-FAK and the downstream p-ERK and p-AKT levels. Furthermore, p-AKT and p-ERK levels were lowered in r-Tinagl1 treated samples that samples treated with either FAK inhibitor 14 (FAKi) and or EGFR inhibitor Erlotinib (Erlo) alone. Moreover, significant difference are not observed between FAKi+Erlo and FAKi+Erlo+r-Tinagl1 in terms of p-EGFR, p-FAK, p-AKT, and especially p-ERK, suggesting r-Tinagl1 exerts its inhibitory effect on oncogenic signaling mostly through blocking EGFR and FAK pathways.

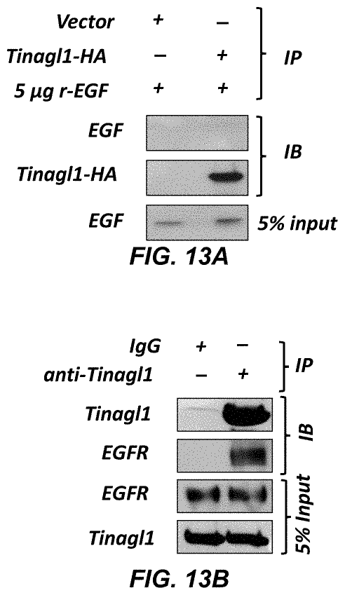

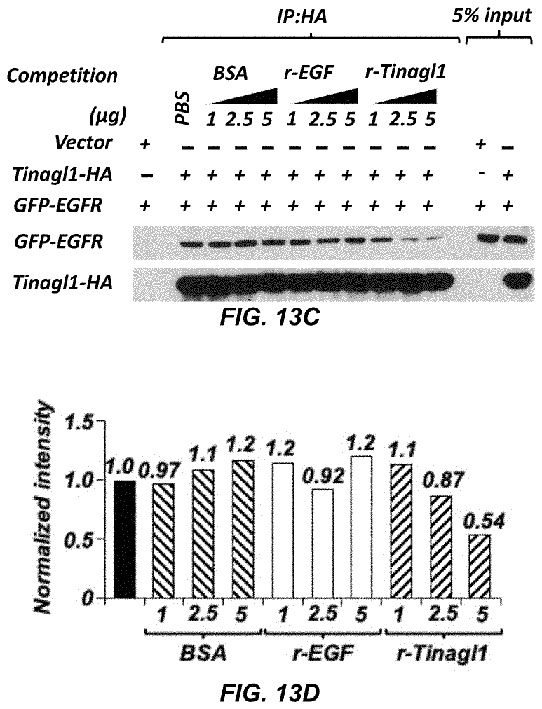

Tinagl1 inhibits EGFR and integrin/FAK signaling pathways with distinct mechanisms. The interaction between EGFR and EGF causes conformational changes in EGFR, leading to EGFR dimerization, phosphorylation, and activation. Previous studies identified an EGFR antagonist, MIF, which competes with EGF for binding to EGFR and subsequently blocks its activation. Whether Tinagl1 inhibits EGFR activation in a similar manner was tested. Referring to FIG. 13A, co-immunoprecipitation experiment failed to detect any interaction between Tinagl1 and EGF, indicating that Tinagl1 does not compete with EGFR for binding to EGF. Whether Tinagl1 and EGF compete with each other to interact with EGFR was tested. Referring to FIG. 13B, immunoprecipitation using recombinant Tinagl1 and EGFR proteins confirmed the direct interaction between these two proteins. However, referring to FIGS. 13C and 13D, recombinant EGF (r-EGF) did not compete with Tinagl1-HA to interact with EGFR. As a positive control, r-Tinagl1 competed with expressed Tinagl1-HA to interact with EGFR. Taken together, these results indicated that Tinagl1 does not inhibit EGFR activation by competing for the same or overlapping EGF binding site.

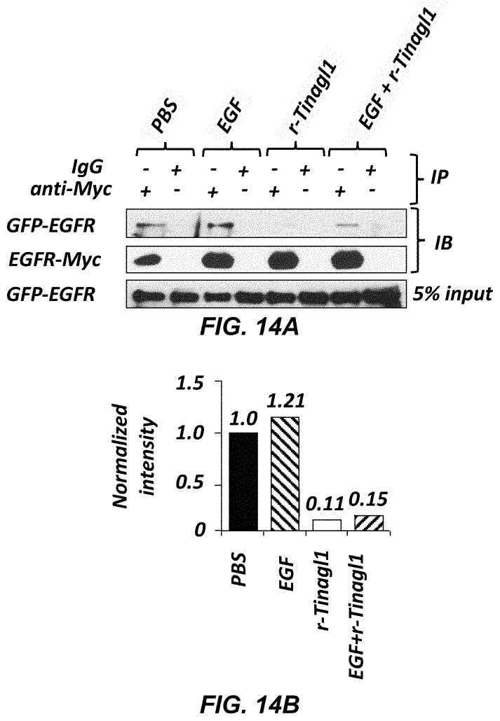

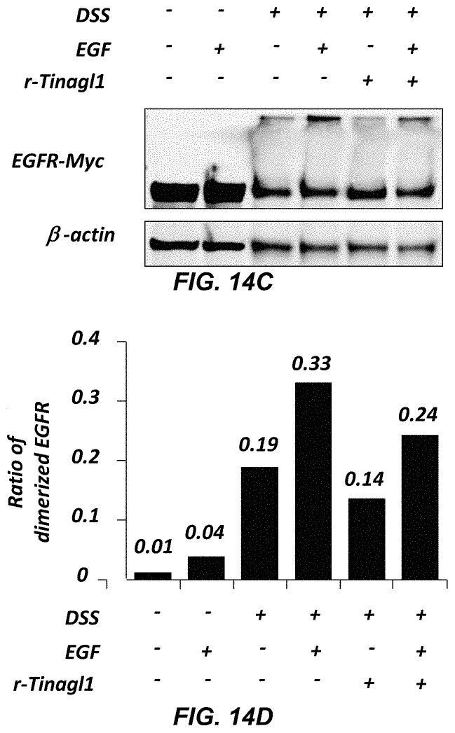

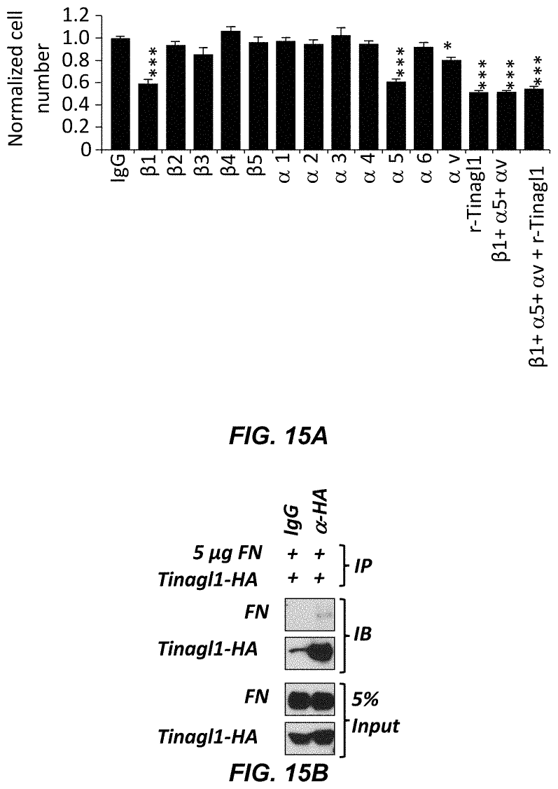

As previously demonstrated, EGFR dimerization is one of the critical steps for its activation after binding to EGF. Whether Tinagl1 prevents EGFR dimerization independent of interfering with EGF binding to EGFR was tested. Referring to FIGS. 14A and 14B, EGFR-GFP and EGFR-Myc were co-expressed in LM2 cells, and IP results indicated that r-Tinagl1 treatment significantly reduced the amount of EGFR-GFP bound by EGFR-Myc in the presence of EGF, suggesting Tinagl1 inhibits EGFR dimerization. To further validate this conclusion, LM2 cells with EGFR-Myc stably expressed were treated with EGF or r-Tinagl1 alone or combination. These cells were then treated with disuccininidylsuberate (DSS) to cross-link the dimerized form of proteins before they were lysed for western blotting analysis.

The assay was performed as previously described (Wang et al., 2015). Briefly, LM2 cells stably expressed EGFR-Myc were seeded in 6-well plates. After 24 hours, the cells were treated with 1 μg/ml of r-Tinagl1 or PBS for 1 hour. Next, the cells were treated with 1 μg/ml of EGF or PBS for another 15 min. The cells were then collected in 0.5 ml PBS. Crosslinking reagent disuccinimidyl suberate (DSS) (ThermoFisher, 21655) were added to a final concentration of 2.5 mM, and the reaction was incubated on ice for 2 hours. The quench solution (1 M Tris-HCl pH 7.5, 1100 dilution) was then added to a final concentration of 10 mM and incubated for 15 min on ice. Finally, the cells were then lysed with IP lysis buffer for 20 min on ice, and EGFR dimerization was analyzed by WB.

Referring to FIGS. 14C and 14D, EGF treatment dramatically increased the dimer form of EGFR, whereas such dimerization was reduced by r-Tinagl1 treatment. Collectively, the data revealed that Tinagl1 inhibits EGFR activation by preventing its dimerization.

Integrin α5β1 and αvβ1 are the major receptors for FN, and the interaction between FN and the integrin receptors triggers the activation of the downstream FAK signal pathway. To test whether Tinagl1 may interfere with cell adhesion mediated by the interaction between FN and its receptors integrin α5β1 and αvβ1, SUM159-M1a cells that were pre-incubated with r-Tinagl1 were seeded on the plates coated with FN or other ECM proteins for various period of time (5 min to 2 hours) and the number of adhered cells were quantified after washing. Tinagl1 blocked cell adhesion mediated by FN but not other ECM proteins such as Laminin and Collagen IV. Next, SUM159-M1a cells that were pre-incubated with r-Tiangl1 or various integrin-blocking antibodies were seeded on the plate coated with FN, and relative numbers of attached cells were measured.

96-wells were coated with 10 μg/ml of indicated proteins. SUM159-M1a cells were preincubated with 10 μg/ml of r-Tinagl1 or BSA for 30 min at 4° C. The cells were then seeded on the plates with 30 k cells per well. The plates were washed with PBS at indicated time points to remove unattached cells. Cells were then lysed, and luciferase activity which represents cell number was measured using the Glomax 96 microplate luminometer (Promega). For antibody blocking assay, 96-well plate was first coated with 10 μg/ml of FN. SUM159-M1a cells were incubated with 10 μg/ml of indicated antibodies (EMD Millipore, ECM430 and ECM440) or r-Tinagl1 at 4° C. for 30 min to block integrin subunits. Cells were then seeded on the plate at 30 k cells per well. 30 min after seeding, the plate was washed with PBS for 5 times and the attached cells were lysed for luciferase assay to determine the cell number.

r-Tinagl1 and blocking antibodies against integrin subunits β1, α5, and av significantly reduced cell adhesion. Referring to FIG. 15A, combining r-Tinagl1 treatment with β1+α5+αv blocking antibodies did not further reduced cell adhesion than either treatment alone, suggesting that Tinagl1 attenuates cell adhesion by blocking the interaction between FN and integrins α5β1 and αvβ1. Moreover, IP-Mass result found Tinagl1 interacted with FN. See FIG. 15B.

Next, a competition IP assay between Tinagl1, FN, and integrin subunits β1, α5, and αv confirmed that Tinagl1 and FN competitively reduced each other's interaction with integrin β1 subunit in a dose-dependent manner (tested at 1, 2.5, and 5 μg). However, Tinagl1 did not compete with the subunits α5 or av for their interaction with FN.

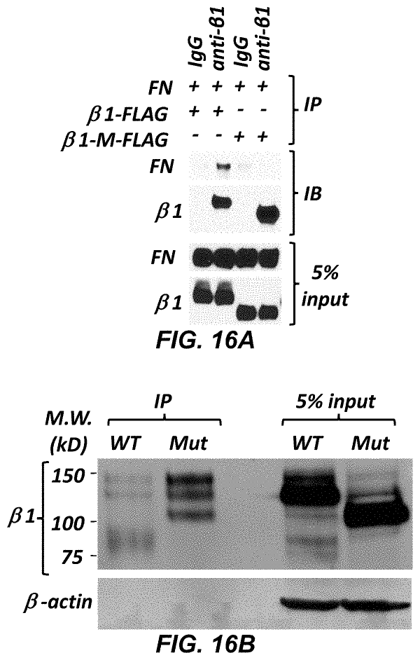

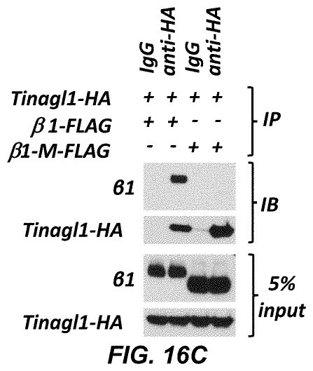

To further confirm that Tinagl1 competes with FN to interact with the β1 subunit [SEQ ID NO.: 23], a mutant integrin β1 subunit (β1-M) with deletion of aa 130-240 was generated, which lack the ability to bind to FN but is still localized to the cell surface (see FIG. 16A, 16B). Referring to FIG. 16C, the mutant β1 subunit also cannot interact with Tinagl1, suggesting that Tinagl1 and FN interact with β1 via the same protein domain. Overall, these data indicated that Tinagl1 competes with FN to interact with integrin β1 subunit, and attenuate integrin/FAK signaling.

Tinagl1 exerts its tumor suppressive function by targeting integrin/FAK and EGFR signaling pathways.

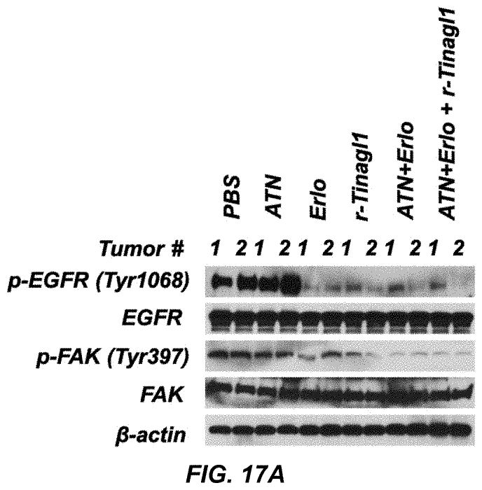

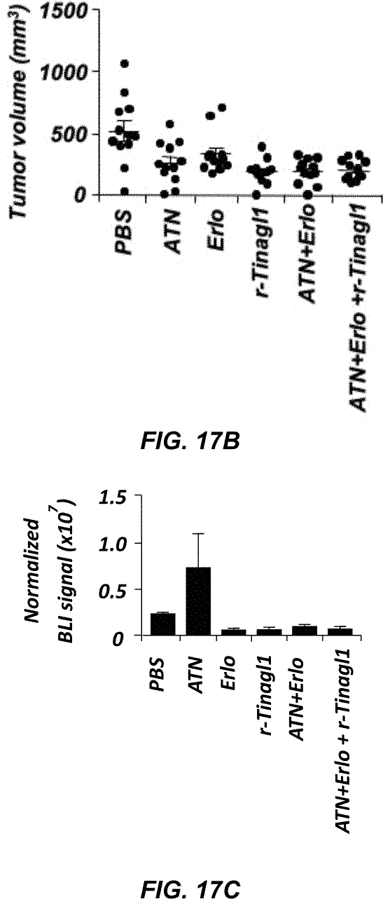

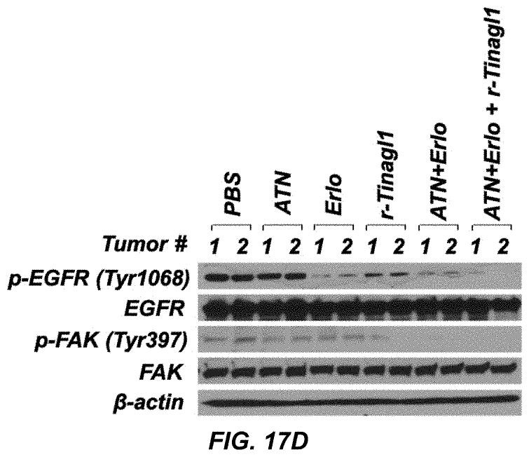

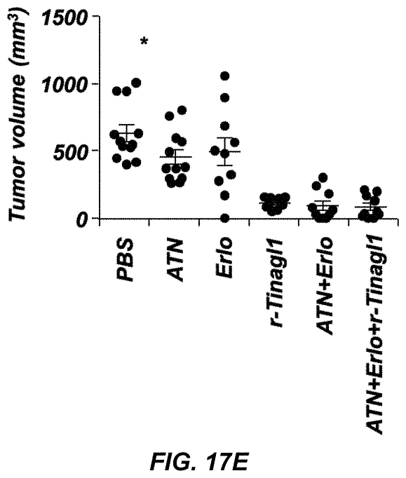

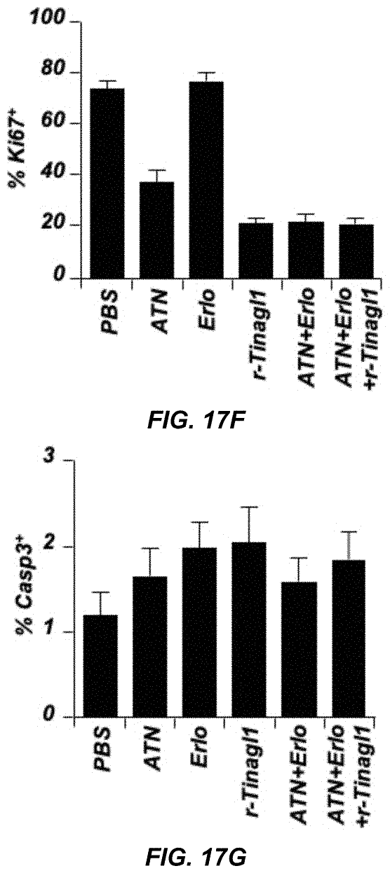

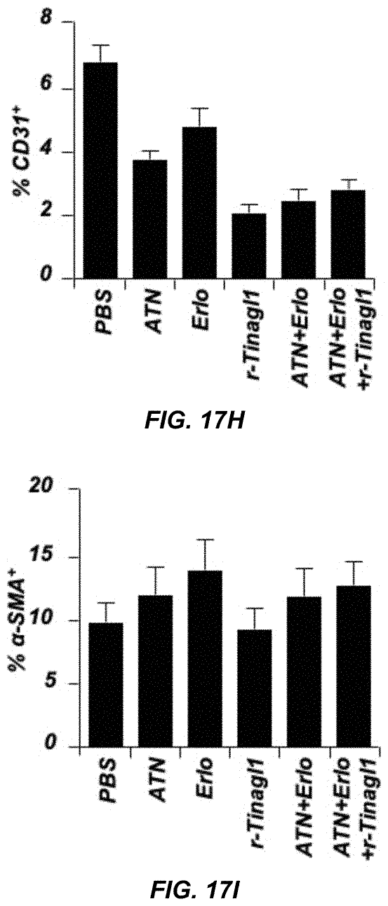

Two molecular targeting reagents, ATN-161 (ATN) and Erlotinib (Erlo) were employed. ATN is an integrin α5β1 antagonist that inhibits the integrin signaling pathway and slows tumor progression. Erlotinib is a well-established small molecular inhibitor of EGFR and is clinically approved for cancer treatment. Mice orthotopically implanted with LM2 cells were split into 6 groups after the tumors reached 2 mm in diameter and subjected to various single or combined agent treatments twice per week [(1) PBS; (2) ATN 30 μg/mouse; (3) Erlo 100 mg/kg; (4) r-Tinagl1 30 μg/mouse; (5) ATN and Erlo; and (6) ATN, Erlo, and r-Tinagl1]. Referring to FIG. 17A, the inhibition of integrin/FAK and EGFR upon each treatment was validated by western blotting and TIC staining.

TIC staining was performed as previously described (Wan et al., 2014). Briefly, Paraffin-embedded primary tumor or lung samples were sliced into 4 μm thickness. The slides were baked overnight at 60° C. Next, the tissue slides were washed with PBS after deparaffinization and hydration and then boiled in citrate buffer at 100° C. for 40 min. After treated with 3% H₂O₂ for 30 min to block endogenous peroxidase, slides were incubated at 4° C. overnight with Ki67 (Leica Biosystem, Ki67-MM1-L-CE-S), cleaved caspase-3 (Cell signaling, 9661S), p-EGFR (Cell signaling, 3777), p-FAK (Cell signaling, 8556), CD31 (Cell signaling, 77699), -SMA (Sigma, A5228), or Tinagl1 (Sigma, HPA048695) antibodies. Following washes with PBS, slides were then incubated with HRP-conjugated goat anti-rabbit or mouse secondary antibody (Genetech) for 30 min at room temperature. Sections were stained by DAB and then counterstained with Gill hematoxylin according to manufacturer's instructions.

To distinguish Tinagl1, p-EGFR, or p-FAK high and low patient samples, two experienced pathologists who were blind to patient status reviewed and scored IHC staining independently, using the staining index (SI), which incorporates intensity and percentage of positive tumor cells. The strength of the staining was scored as follows: 0, no staining; 1, weak; 2, moderate; 3, strong; and the percentage of cells stained was scored as follows: 0, no staining; 1, <10%; 2, 10-50%; and 3, >50% tumor cells. If there was a disagreement between the two pathologists, a third pathologist was consulted to reach a consensus. The SI was derived by multiplying the staining score and percentage score. Samples with SI greater than 4 were considered as Tinagl1, p-EGFR, or p-FAK high expression.

Referring to FIG. 17B, results indicated that Erlotinib treatment alone trended toward reducing primary tumor growth but did not reach statistical significance, supporting the possibility of compensatory networks, such as integrin/FAK, that mediate the escape from EGFR inhibition. Further supporting this notion, combined treatment using ATN+Erlotinib, or r-Tinagl1, which target both EGFR and integrin pathways, significantly suppressed primary tumor growth. Notably, combing all three agents did not produce further increase of therapeutic benefit than using Tinagl1 alone.

Referring to FIG. 17C, erlotinib or r-Tinagl1 treatment reduced spontaneous lung metastasis of LM2 tumors while no reduction was observed by ATN treatment alone. In the SUM159-M1a model, while Erlotinib alone can reduced lung metastasis, dual inhibition of EGFR and integrin/FAK by ATN+Erlotinib or r-Tinagl1 had a stronger effect than single treatment of Erlotinib in reducing lung metastasis. See FIGS. 17D and 17E. Collectively, the data suggested EGFR and integrin signaling might compensate each other to promote TNBC progression, and Tinagl1 exerted its tumor inhibitory function by simultaneously targeting both integrin and EGFR signaling pathways.

Referring to FIGS. 17F-17I, consistent with observation of in vivo treatment response, Ki67 staining tumor samples revealed that ATN suppressed proliferation in primary tumor but not lung metastatic nodules. In contrast, Erlo inhibited tumor cell proliferation in lung metastatic nodules but not primary sites. Both ATN+Erlo and r-Tinagl1 treatments has strong anti-proliferative effects on both primary tumors and lung metastases. No difference was observed between ATN+Erlo and ATN+Erlo+r-Tinagl1 groups, suggesting that Tinagl1 inhibits tumor proliferation mostly through targeting EGFR and integrin/FAK pathways. While no difference in apoptotic activity and tumor-associated fibroblast infiltration was observed, CD31⁺ endothelium was significantly reduced upon erlotinib or r-Tinagl1 treatments in both primary and lung metastasis, indicating potentially additional anti-tumor effects of r-Tinagl1 through reducing angiogenesis.

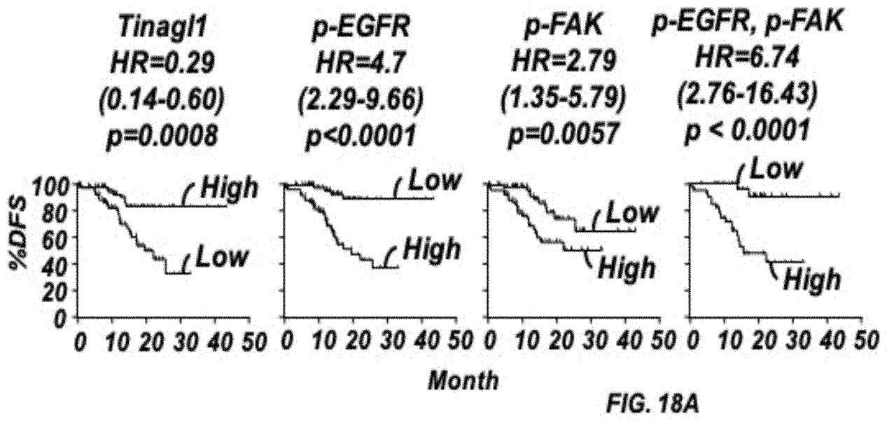

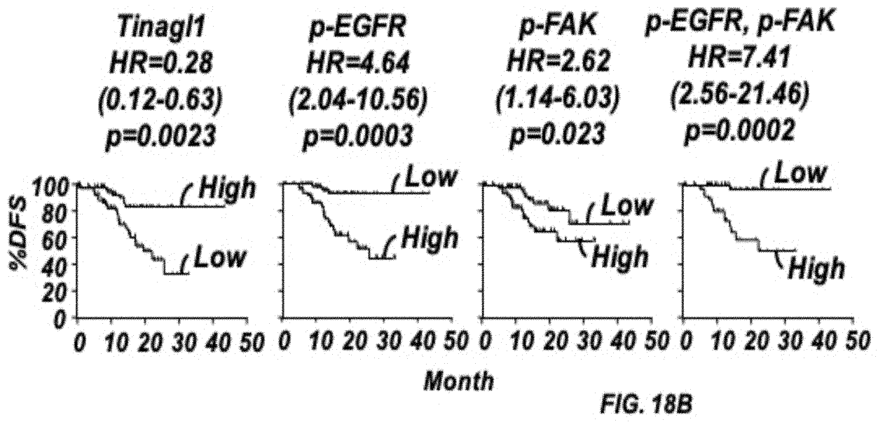

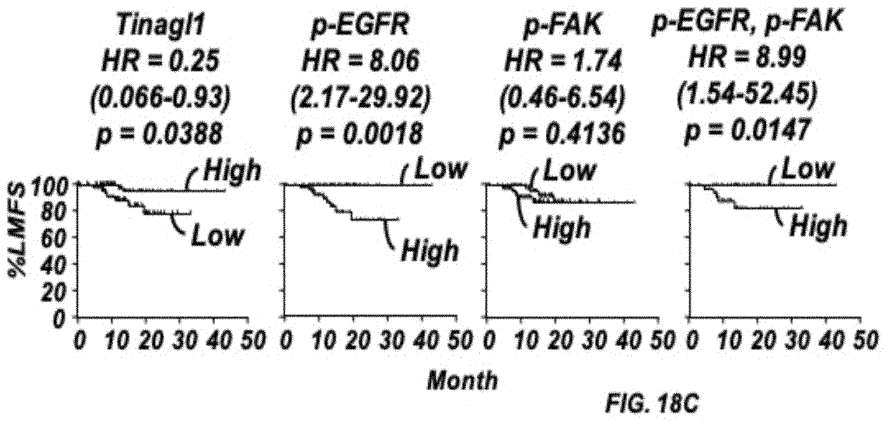

Tinagl1 is negatively correlated with EGFR and FAK activation in TNBC patient samples. IHC staining of the primary tumors indicated that Tinagl1 protein levels were negatively correlated with the activation status of EGFR and FAK in TNBC patients. Similarly, Tinagl1 protein levels were found to be negatively correlated with the activation of both EGFR and FAK in metastatic lung samples. Referring to FIG. 18A, consistent with the previously discussed mRNA-based analysis, high Tinagl1 protein levels correlated with better DFS, whereas high activation of EGFR or/and FAK is linked to advanced tumor stages and worse survival. Referring to FIG. 18B, consistently, when analyzing distant metastasis free survival (DMFS) of patients, high Tinagl1 protein levels correlated with good prognosis, while the high activation of EGFR or/and FAK correlated with poor prognosis. Referring to FIG. 18C, interestingly, there was no significant correlation between the FAK activation and lung metastasis-free survival (LMFS), which is in line with the mouse treatment result showing that targeting integrins alone does not reduce lung metastasis. Multivariable Cox analysis further indicated that low Tinagl1 and high p-EGFR levels are strongly linked to poor DFS, with p-FAK also showing a strong hazard ratio. These findings suggest that Tinagl1 regulation of EGFR and integrin/FAK activation play a functional role in regulating the disease progression of cancers such as TNBC.

Thus, in some embodiments, the patient may have previously been diagnosed with a cancer having active Integrin signaling, active EGFR signaling, or a combination thereof. As is understood in the art, drugs that inhibitor EGFR signaling have been used to treat at least, for example, colorectal cancer, head and neck cancers, non-small cell lunch cancer (NSCLC), and pancreatic cancer. See Seshacharyulu et al., “Targeting the EGFR signaling pathway in cancer therapy”, Expert OpinTher Targets. 2012 January; 16(1): 15-31; Ciardiello et al., “EGFR antagonists in cancer treatment”, N Engl J Med 2018 Mar. 13; 358; 11 1160-75. Integrin signaling is involved in resistance to therapies targeting growth factor receptors in many cancer types, and thus play a role in, e.g., head and neck squamous cell carcinoma tumors, pancreatic cancer tumors, colon cancer tumors, lung cancer tumors, glioma tumors, breast cancer tumors, acute myeloid leukemia tumors, hepatic cancer tumors, gastric cancers, See Cruz da Silva et al., “Role of Integrins in Resistance to Therapies Targeting Growth Factor Receptors in Cancer”, Cancers 2019, 11, 692. As fragments of Tinagl1 can function as an inhibitor of both EGFR and Integrin signaling, the fragments of Tinagl1 can be used as a therapeutic for the above-referenced cancers.

Stable or inducible ectopic expression of Tinagl1 in cancer cells inhibit tumor growth and, e.g., lung metastasis. Importantly, recombinant Tinagl1 protein treatment in mice suppressed tumor progression without causing significant toxicity in animals, indicating a therapeutic application of Tinagl1.

In some embodiments, the inhibitor (comprising or consisting of the first 94 amino acids of a Tinagl1 protein) are utilized with a pharmaceutically acceptable carrier.

Pharmaceutically acceptable carriers that can be used in the composition may include any substance that can effectively retain the inhibitor in a dispersed state in a final solid dosage form. Suitable pharmaceutically acceptable carriers include, for example, pharmaceutically acceptable polymers and pharmaceutically acceptable ureas. Preferred carriers include polyethylene glycols (e.g., PEG 1000, PEG 1500, PEG 3350, PEG 4600, PEG 6000 and PEG 8000), polyvinylpyrrolidones (e.g., Kollidon 12 PF, Kollidon 17 PF, Kollidon 25 PF, Kollidon 30 PF, Kollidon 90 PF etc.), polyvinylalcohols, cellulose derivatives (e.g., hydroxypropylmethylcellulose (HPMC), hydroxypropylcellulose (HPC)), polyacrylates, polymethacrylates, polyglycolyzed glycerides, ureas, sugars (e.g., lactose), polyols, and mixtures thereof. The best carrier to be used for a particular composition will depend on a variety of factors including the other ingredients in the composition and the specific method to be employed in the preparation of the composition. The amount of pharmaceutically acceptable carrier may vary over a wide range and the optimum amount for a particular composition will again depend on the other ingredients in the composition and the method of preparation to be employed and can be easily determined by the skilled pharmaceutical technician. In general, however, the pharmaceutically acceptable carrier may be present in the solid dispersion composition in an amount up from about 1 to 99% by weight.

The inhibitor may be administered in any technique known to those of skill in the art, including but not limited to intravenously, subcutaneously, intramuscularly, intralesionally, intraperitoneally, via liposomes, transmucosally, intestinally, topically, via nasal route, orally, via anal route, via ocular route, or via otic route.

Tinagl1 interacts with EGFR and prevents its ligand-induced dimerization and receptor activation. Tinagl1 also interacts with various integrin α5β1 and αvβ1. Tinagl1 also suppresses FN-induced integrin/FAK signaling. By inhibiting integrin/FAK and EGFR signaling pathway simultaneously without significant side effects and toxicities that are often observed in single or combined treatment of tyrosine kinase inhibitors, Tinagl1 represents a potentially new strategy of targeting oncogenic pathways using ECM component proteins.

While there is a clear trend that Tinagl1 is also correlated with good prognosis in ER/PR⁺ and HER2⁺ subtypes, the correlation is not as strong as in TNBC and is not statistically significant. This could be due to the following two reasons: First, tumor progression of ER/PR⁺ and HER2⁺ subtypes is predominantly influenced by the estrogen receptor and HER2 pathways respectively, which are not the major targets of Tinagl1. Second, EGFR and ITGB1 are highly expressed in TNBC tumors and expression levels of EGFR and integrin β1 subunit correlated with poor clinical outcome and progression in TNBC. This suggests that EGFR and integrins α5β1 and αvβ1 may play critical roles in promoting TNBC progression. As Tinagl1 targets EGFR and integrin/FAK pathways via interacting with EGFR and β1, α5, αv subunits, all these facts may contribute to the observation that Tinagl1 has more significant clinical importance in TNBC patients. Nevertheless, the inhibitory effect of Tinagl1 in breast cancer progress is not limited to TNBC. instead, Tinagl1 may have a universal effect on the cancers, such as breast cancers, that are driven by EGFR and integrin/FAK signaling rather having a more restrictive role on TNBC. It has been reported that early stages of PyMT-induced tumor mimic luminal B subtype of human breast cancer, which is ER/PR⁺. The tumors lose ER/PR expression when they progress to late stages. Moreover, previous studies indicated that EGFR and integrin/FAK pathways are critical for PyMT tumor progression. The disclosed results demonstrate that Tinagl1-KO promoted PyMT tumor initiation at early stages, and enhanced tumor growth and lung metastasis at later stages. These results further support the notion that Tinagl1 does not selectively suppress TNBC only; instead, it may also have inhibitory effects on other subtypes which are driven by EGFR and integrin/FAK signaling.

In some embodiments, when treating a patient, at least one additional therapeutic agent is administered to the patient. The additional therapeutic agent may be a chemotherapeutic agent, an anti-cell proliferation agent, a gene therapy agent, an immunotherapy agent, an antibody-drug conjugate, an antibody-toxin conjugate, and/or an immune checkpoint inhibitor.

Therapeutic agents include, but are not limited to, alkylating agents, alkyl sulfonates, aziridines, ethylenimines, methylamelamines, colchicines, camptothecins, nitrogen mustards, nitrosoureas, plant alkaloids, bisphosphonates, anthracyclines, anti-metabolites, anti-microtubule agents, topoisomerase inhibitors, cytotoxic antibiotics, metal salts, toxoids, taxanes, pyrimidine analogs, purine analogs, aromatase inhibitors, mitomycins, androgens, anti-adrenals, folic acid replenishers, anti-folates, dihydrofolate reductase inhibitors, thymidylate synthase inhibitors, vinca alkaloids, and anti-hormonal agents, as well as pharmaceutically acceptable salts, acids, or derivatives of any of the above, as well as combinations of two or more of the above.

Chemotherapeutic agents include, but are not limited to, TAXOL® (paclitaxel), docetaxel, ADRIAMYCIN® (doxorubicin), epirubicin, 5-fluorouracil, CYTOXAN® (cyclophosphamide), carboplatin, PLATINOL® (cisplatin), IBRANCE® (palbociclib), ARIMIDEX® (anastrozole), XELODA® (capecitabine), DOXIL® (doxorubicin liposomal injection), AROMASIN® (exemestane), GEMZAR® (gemcitabine), IXEMPRA® (ixabepilone), and FEMARA® (letrozole).

Anti-cell proliferation agents include, but are not limited to, nucleotide and nucleoside analogs, such as 2-chloro-deoxyadenosine, adjunct antineoplastic agents, alkylating agents, nitrogen mustards, nitrosoureas, antibiotics, antimetabolites, hormonal agonists/antagonists, androgens, antiandrogens, antiestrogens, estrogen & nitrogen mustard combinations, gonadotropin releasing hotmone (GNRH) analogues, progestrins, immunomodulators, miscellaneous antineoplastics, photosensitizing agents, and skin & mucous membrane agents.

Gene therapy agents include, but are not limited to, a solution, mixture, or other formulation containing a polynucleotide to be delivered intracellularly. A transfection agent usually includes a carrier polynucleotide, termed “expression vector,” also known as “gene delivery vector,” linked to a transgene and, optionally, other compounds that may facilitate the transfer of the polynucleotide across the cell wall. Typically, such compounds reduce the electrostatic charge of the cell surface and the polynucleotide itself or increase the permeability of the cell wall. Examples include cationic liposomes, calcium phosphate, polylysine, vascular endothelial growth factor (VEGF), etc. Hypertonic solutions, containing, for example, NaCl, sugars, or polyols, can also be used to increase the extracellular osmotic pressure thereby increasing transfection efficiency. The gene therapy solutions may also include enzymes such as proteases and lipases, mild detergents and other compounds that increase permeability of cell membranes. The methods of the invention are not limited to any particular composition of the transfection agent and can be practiced with any suitable agent so long as it is not toxic to the subject or its toxicity is within acceptable limits.

Immunotherapy agents include, but are not limited to, a cancer vaccine, hormone, epitope, cytokine, tumor antigen, CD4 cell stimulator, NKT cell agonist, or adjuvant. For example, the immunotherapeutic agent can be an interferon, interleukin, tumor necrosis factor, ovalabumin, Neuvenge®, Oncophage, CimaVax-EGF, Mobilan, α-Gal glycolipid, α-Galactosylceramide (α-GalCer), β-mannosylceramide (β-ManCer), adenovirus delivered vaccines, Celldex's CDX1307 and CDX1401; GRNVAC1, viral based vaccines, MVA-BN, PROSTVAC®, Advaxis′; ADXS11-001, ADXS31-001, ADXS31-164, BiovaxID, folate binding protein (E39), Granulocyte macrophage colony stimulating factor (GM-CSF) with and without E75 (NeuVax) or OncoVEX, trastuzumab, Ae-37, IMA901, SC1B1, Stimuvax, peptides that can elicit cytotoxic lymphocyte response, peptide vaccines including telomerase peptide vaccine (GV1001), survivin peptide, MUC1 peptide, ras peptide, TARP 29-37-9V Peptide epitope enhanced peptide, DNA Vector pPRA-PSM with synthetic peptides E-PRA and E-PSM; Ad.p53 DC vaccine, NY-ESO-1 Plasmid DNA (pPJV7611), genetically modified allogeneic (human) tumor cells for the expression of IL-1, IL-7, GM-CSF, CD80 or CD154, HyperAcute®-Pancreatic cancer vaccine (HAPa-1 and HAPa-2 components), Melaxin (autologous dendritoma vaccine) and BCG, GVAX (CG8123), CD40 ligand and IL-2 gene modified autologous skin fibroblasts and tumor cells, ALVAC-hB7.1, VaximmGmbh's VXMO1, Immunovative Therapies' AlloStim-7, ProstAtak™, TG4023 (MVA-FCU1), Antigenic's HSPPC-96, Immunovaccine Technologies' DPX-0907 which consists of specific HLA-A2-restricted peptides, a universal T Helper peptide, a polynucleotide adjuvant, a liposome and Montanide (ISA51 VG), GSK2302032A, Memgen's ISF35, Avax'sOVax: Autologous, DNP-Modified Ovarian vaccine, Theratope®, Ad100-gp96Ig-HLA A1, Bioven's recombinant Human rEGF-P64K/Montanide vaccine, TARP 29-37, or Dendreon's DN24-02.

Other additional treatments that can be utilized include anti-angiogenic agents (such as AVASTIN® (bevacizumab)), and HER2+ targeted therapy agent (such as HERCEPTIN® (trastuzumab)).

Antibody-drug conjugates (ADCs) refer to molecules comprising an antigen binding protein that is linked or otherwise joined, usually via a chemical linkage, to a drug molecule/protein. Non-limiting examples of such ADCs include: Trastuzumab emtansine (T-DM1, Kadcyla), Brentuximab vedotin (SGN-35), Inotuzumab ozogamicin (CMC-544), Pinatuzumab vedotin (RG-7593), Polatuzumab vedotin (RG-7596), Lifastuzumab vedotin (DNIB0600A, RG-7599), Glembatuzumab vedotin (CDX-011), Coltuximab ravtansine (SAR3419), Lorvotuzumab mertansine (IMGN-901), Indatuximab ravtansine (BT-062), Sacitizumab govitican (INMU-132), Labetuzumab govitican (INMU-130), Milatuzumab doxorubicin (IMMU-110), Indusatumab vedotin (MLN-0264), Vadastuximab talirine (SGN-CD33A), Denintuzumab mafodotin (SGN-CD19A), Enfortumab vedotin (ASG-22ME), Rovalpituzumab tesirine (SC16LD6.5), Vandortuzumab vedotin (DSTP3086S, RG7450), Mirvetuximab soravtansine (IMGN853), ABT-414, IMGN289, or AMG595.

Antibody-toxin conjugates (immunotoxins) refer to molecules comprising an antigen binding protein that is linked or otherwise joined, usually via a chemical linkage, to a cytotoxin moiety, such as a protein toxin. Non-limiting examples of antibody toxin conjugates include: MH3-Bl/rGel, denileukin diftitox (DAB389IL2), moxetumomab pasudotox (CAT-8015), oportuzumab monotox (VB4-845), Resimmune, LMB-2, DT2219ARL, HuM195/rGel, RG7787, MOC31PE or D2C7-IT.

Immune checkpoint inhibitors (ICIs) refer to molecules that totally or partially reduce, inhibit, interfere with or modulate one or more checkpoint proteins (proteins that regulate T-cell activation or function). Numerous checkpoint proteins are known, such as CTLA-4 and its ligands CD80 and CD86; and PD1 with its ligands PDL1 and PDL2. These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies. Non-limiting examples of immune checkpoint inhibitors include anti-PD1 antibodies and anti-PDL1 antibodies.

As used herein, the term “antibody” includes reference to both glycosylated and non-glycosylated immunoglobulins of any isotype or subclass or to an antigen-binding region thereof that competes with the intact antibody for specific binding, unless otherwise specified, including monoclonal antibodies, bispecific antibodies, minibodies, domain antibodies, synthetic antibodies, antibody mimetics, chimeric antibodies, humanized antibodies, human antibodies, antibody fusions, antibody conjugates, single chain antibodies, antibody derivatives, antibody analogues and fragments thereof, respectively. Also included are immunological fragments of an antibody (e.g., a Fab, a Fab′, a F(ab′)₂, or a scFv), irrespective of whether such antibodies are produced, in whole or in part, via immunization, through recombinant technology, by way of in vitro synthetic means, or otherwise. Thus, the term “antibody” is inclusive of those that are prepared, expressed, created or isolated by recombinant means, such as (a) antibodies isolated from an animal (e.g., a mouse) that is transgenic for human immunoglobulin genes or a hybridoma prepared therefrom, (b) antibodies isolated from a host cell transfected to express the antibody, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences. Such antibodies have variable and constant regions derived from germline immunoglobulin sequences of two distinct species of animals. In certain embodiments, however, such antibodies can be subjected to in vitro mutagenesis (or, when an animal transgenic for human immunoglobulin sequences is used, in vivo somatic mutagenesis) and thus the amino acid sequences of the V_{H} and V_{L} regions of the antibodies are sequences that, while derived from and related to the germline V_{H} and V_{L} sequences of a particular species (e.g., human), may not naturally exist within that species' antibody germline repertoire in vivo. Unless otherwise indicated, the term “antibody” includes, in addition to antibodies comprising two full-length heavy chains and two full-length light chains, derivatives, variants, fragments, and muteins thereof. In some instances, “antibody” may include fewer chains such as antibodies naturally occurring in camelids which may comprise only heavy chains.

In some embodiments, more precise, targeted therapy techniques based on the one or more fragments of Tinagl1 (such as, e.g., the first 94 amino acids of Tinagl1) are used to improve efficacy and increase the therapeutic window by, e.g., reducing systemic toxicity. Non-limiting examples of the targeted therapy techniques include antibody conjugation of the Tinagl1 fragment, creation of fusion proteins using the Tinagl1 fragment, chemical modification of the Tinagl1 fragment, or mutation of the Tinagl1 fragment.

For example, the disclosed proteins may be useful as active ingredients (immunogens) in immunogenic compositions, and such compositions may be useful as vaccines. Vaccines according to the invention may either be prophylactic (i.e., to prevent infection) or therapeutic (i.e., to treat infection). Immunogenic compositions will be pharmaceutically acceptable. They will usually include components in addition to the antigens e.g. they typically include one or more pharmaceutical carrier(s), excipient(s) and/or adjuvant(s). Also disclosed is a vaccine comprising a nucleic acid sequence encoding a fusion protein comprising one or more alpha virus surface membrane glycoprotein operatively linked to one or more tumor associated antigen. The vaccine may thus comprise a nucleic acid construct or comprises a fusion protein as defined above. The vaccine may furthermore be used as a medicament.

The vaccine composition can be formulated according to known methods such as by the admixture of one or more pharmaceutically acceptable carriers, also known as excipients or stabilizers with the active agent. These excipients may be acceptable for administration to a subject, preferably to vertebrates and more preferably to humans as they are non-toxic to the cell or individual being exposed thereto at the dosages and concentrations employed. In certain embodiments, an acceptable carrier is an aqueous pH buffered solution. Examples of such excipients, carriers and formulation may be found in, e.g., Remington's Pharmaceutical Sciences (Maack Publishing Co, Easton, Pa.). Examples of physiologically acceptable carriers include but are not limited to: buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polyethylene glycol (PEG).

To formulate a pharmaceutically acceptable composition suitable for effective administration, such compositions will contain an effective amount of Tinagl1 or fragment thereof as described above, within a delivery vehicle or the fusion protein as described herein. A carrier may be used as a scaffold by coupling the fusion proteins to improve the induction of an immune response. The carrier protein may be any conventional carrier including any protein suitable for presenting immunogenic determinants. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Immunization of the animal may be carried out with adjuvants and/or pharmaceutical carriers. Conventional carrier proteins include, but are not limited to, keyhole limpet hemocyanin, serum proteins such as transferrin, bovine serum albumin, or human serum albumin, an ovalbumin, immunoglobulins, or hormones, such as insulin. The carrier may be present together with an adjuvant. Vaccine compositions are useful for prophylactic and therapeutic use, including stimulating an immune response in a subject. The vaccine composition disclosed herein does not induce any systemic or local toxicity reactions or any other side effects. Adjuvants may be included in the vaccine composition to enhance the specific immune response. Thus, it is particularly important to identify an adjuvant that when combined with the antigen(s)/nucleic acid constructs and/or delivery vehicles (any of which may also be referred to as immunogenic determinant), results in a vaccine composition capable of inducing a strong specific immunological response. The immunogenic determinant may also be mixed with two or more different adjuvants prior to immunization. A large number of adjuvants have been described and used for the generation of antibodies in laboratory animals, such as mouse, rats and rabbits. In such setting the tolerance of side effect is rather high as the main aim is to obtain a strong antibody response.

Immunogenic compositions may also contain diluents such as buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with non-specific serum albumin are exemplary appropriate diluents.

The pH of a composition will generally be between 5.0 and 8.1, and more typically between 6.0 and 8.0 e.g. 6.5 and 7.5, or between 7.0 and 7.8. The composition is preferably sterile. The composition is preferably non-pyrogenic e.g. containing <1 EU (endotoxin unit, a standard measure) per dose, and preferably <β1 EU per dose. The composition is preferably gluten free.

Some treatment methods may also include administering ionizing radiation to the patient.

In some treatment regimens, the patient may not be administered any cancer therapeutic agent except the inhibitor. Alternatively, in some embodiments, the patient is administered an additional therapeutically effective amount of the inhibitor at a second point in time after the therapeutically effective amount of the inhibitor was first administered. For example, in some embodiments, additional treatments are given multiple times a day, once a day for a week, a month, or multiple months, or once a week for multiple weeks.

The above-referenced human breast cancer cell lines MDA-MB-231 and LM2, HEK293T, and mouse breast cancer cell lines, FVB-MMTV-PyMT, 67NR, 4TO7, 4T1, 168FARN, and 66c14 were grown in DMEM supplemented with 10% FBS and pen/strep. Retrovirus-producing cells line H29 was grown in the same media supplemented with 2 μg/ml puromycin, 300 μg/ml G418 and 1 μg/ml doxycycline. SUM159-M1a cells were culture with F12 media supplemented with 10% FBS, 10 μg/ml Insulin, 20 μg/ml EGF and pen/strep. HCC1937 cells were grown in RPMI-1640 supplemented with 10% FBS and pen/strep.

For xenograft studies, 8-weeks immunocompromised NOD Scid Gamma (NSG) or immunocompetent FVB females were used. Cells were suspended in 10 μl of PBS for mammary gland injection (MFP), or were suspended in 100 μl of PBS for intravenous injection. For human patient-derived xenograft (PDX, HCL001, kindly provided by Dr. Alana Welm) study, we followed the standard protocol for PDX transplantation, maintenance and digestion of the tumors (DeRose et al., 2011). 2×10⁴ cells were suspended in 10 μl of PBS and subjected to MFP injection. For recombinant Tinagl1 (r-Tinagl1), ATN-161, Erlotinib treatments, the mice were injected with 30 μg/mouse, 30 μg/mouse, and 100 mg/kg respectively twice per week via tail-vein. Primary tumors were quantified once per week via caliper measurement. All cell lines used for lung metastasis experiments were stably labeled with a firefly luciferase expressing vector and were monitored by weekly bioluminescent imaging (BLI). At protocol-defined endpoints, lungs were dissected and fixed in bouin's solution and the metastatic lesions were counted.

Also disclosed is a gene therapy treatment. As understood by those of skill in the art, gene therapy is the process of introducing foreign genomic materials into host cells to elicit a therapeutic benefit. Somatic gene therapy involves the insertion of genes into diploid cells of an individual where the genetic material is not passed on to its progeny. As understood by those of skill in the art, there are three general types of somatic gene therapy: ex vivo delivery, in situ delivery, and in vivo delivery. In ex vivo delivery, the genetic material is removed from the target tissue or bone marrow, cultivated and manipulated in vitro, and then transducted and/or transfected into the target tissue. For in situ delivery, the genetic material is administered directly into the target tissue. For in vivo delivery, the genetic material is transferred into the target tissue through an appropriate vector (e.g., viral or non-viral).

Viral vectors. All viral vector genomes have been modified by deleting some areas of their genomes so that their replication becomes deranged. As known to those of skill in the art, numerous viral vectors are in common usage, including retroviral vectors (including lentiviral vectors), adenoviral vectors (e.g., adenovirus type 2 and 5 serotypes), adeno-associated vectors (AAVs) (e.g., AAV1, AAV2, AAV5, AAV6, AAV8, and AAV9), helper-dependent adenoviral vectors, Hybrid adenoviral vectors, and Herpes simplex virus (HSV) vectors.

Non-viral delivery systems. Nonviral systems comprise all the physical and chemical systems except viral systems and generally include either (i) chemical methods, such as the use of nanomeric complexes including lipoplexes (complex between cationic liposome or micelle and nucleic acids) and polyplexes (complex between cationic polymer and nucleic acid) and delivery by cationic particles, or (ii) physical methods, such as providing naked DNA, DNA particle bombardant via gene gun, electroporation, hydrodynamic delivery, ultrasound utilization, and magnetofection.

Cationic systems are general comprised of either a single synthetic cationic amphiphile (cytofectin), such as DOTAP, DOTMA, DOSPA, DOGS, or more commonly of a combination of a cationic amphiphile and a neutral lipid, such as DOPE and cholesterol. Cationic liposome-mediated delivery of DNA materials is generally preferred in vivo when the mol ratio of cationic liposome to nucleic acid in the lipoplex mixture is such that the positive/negative charge ratio is around 1 or greater and in vitro the optimal ratio is closer to 1. However, multivalent lipids with long and unsaturated hydrocarbon chains are more efficient than monovalent cationic lipids with the same hydrophobic chains. For gene transfer in vivo, non-limiting examples include Chol/DOPE (1:1), DOTMA/DOPE (1:1), and DOTAP/DOPE (1:1).

Non limiting examples of cationic polymers include poly-l-lysine (PLL) and polyethylenimine (PEI). PLL, and PLL with PEG attached to the polymer, has been used in a variety of polymerizations of lysine ranging from 19 to 1116 amino acid residues (3.97-233.2 kDa). While the molecular weight of the polymer increases, the net positive charge of it also increases and are therefore able to bind DNA tighter and form more stable complexes. There is a relationship between the length of the polymer, gene delivery efficiency, and toxicity as the length of the polymer increases, so does its efficiency and its toxicity. As known to those of skill in the art, different homogenous PLL-conjugated peptides have been developed that have low toxicity, higher efficiency, and site-specific attachment of ligands used for cell targeting. One preferred peptide sequence contains 18 lysines followed by a tryptophan and alkylated cysteine (AlkCWK18). Conjugation of some agents, such as galactose, anti-CD3 antibodies and RGD motif-containing peptides can facilitate polyplex cellular uptake.

The disclosed gene therapy treatment provides for the delivery of the Tinagl1 protein or a fragment thereof (such as the first 94 amino acids of Tingal1), any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein for treating a cancer in a subject. The method includes administering to a subject a pharmaceutical composition comprising a gene under control of a promoter sequence, the gene capable of expressing at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein, where the gene is delivered by a viral or non-viral delivery system.

1. A method of treating cancer comprising administering to a patient in need thereof a therapeutically effective amount of an inhibitor of the epidermal growth factor receptor (EGFR) pathway and the integrin/focal adhesion kinase (FAK) pathway, wherein the inhibitor comprises at least the first 94 amino acids of a Tinagl1 protein [SEQ ID NO.: 1], any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein.

2. The method according to claim 1, wherein the inhibitor interacts with EGFR, integrin α5β1, αvβ1, or a combination thereof.

3. The method according to claim 1, wherein the patient is a mammal.

4. The method according to claim 1, wherein the patient is a human.

5. The method according to claim 1, wherein the patient is a female human.

6. The method according to claim 1, wherein the patient has previously been diagnosed with triple negative breast cancer.

7. The method according to claim 1, wherein the patient has previously been diagnosed with a cancer having active Integrin signaling, active EGFR signaling, or active Integrin and EGFR signaling.

8. The method according to claim 1, wherein the Tinagl1 protein is human Tinagl1 protein [SEQ ID NO.: 1].

9. The method according to claim 1, wherein the at least first 94 amino acids of the human Tinagl1 protein is produced by recombinant or endogenous expression.

10. The method according to claim 1, further comprising extracting the at least first 94 amino acids of the human Tinagl1 protein from a native source selected from the group consisting of mammalian cell cultures, tissues or bodily fluids.

11. The method according to claim 1, wherein the human Tinagl1 protein is produced by recombinant expression in mammalian, insect, bacterial, or yeast cells.

12. The method according to claim 1, wherein the human Tinagl1 protein is produced by recombinant expression in E. coli, CHO cells, or HEK cells.

13. The method according to claim 1, further comprising administering to the patient at least one additional therapeutic agent selected from the group consisting of a chemotherapeutic agent, an anti-cell proliferation agent, a gene therapy agent, an immunotherapy agent, an antibody-drug conjugate, an antibody-toxin conjugate, and an immune checkpoint inhibitor.

14. The method according to claim 1, wherein the patient is not administered any cancer therapeutic agent except Tinagl1 protein.

15. The method according to claim 1, further comprising administering ionizing radiation to the patient.

16. The method according to claim 1, wherein the Tinagl1 protein is administered intravenously, subcutaneously, intramuscularly, intralesionally, intraperitoneally, via liposomes, transmucosally, intestinally, topically, via nasal route, orally, via anal route, via ocular route, or via otic route.

17. The method according to claim 1, further comprising administering to the patient an additional therapeutically effective amount of the inhibitor at a second point in time after the therapeutically effective amount of the inhibitor was first administered.

18. The method according to claim 1, further comprising determining an expression level of a Tingal1 gene or of a Tingal1 protein or a variant thereof of the subject.

19. An isolated recombinant protein, comprising the first 94 amino acids of a Tinagl1 protein.

20. The isolated recombinant protein according to claim 19, wherein the protein is a full length Tinagl1 protein.

21. The isolated recombinant protein according to claim 19, wherein the protein is the human Tinagl1 protein [SEQ ID NO.: 1].

22. A therapeutic dose, comprising the isolated recombinant protein according to claim 19 and a pharmaceutically acceptable carrier.

23. A method for treating a cancer in a subject via gene therapy, comprising the steps of:
administering to a subject a pharmaceutical composition comprising a viral or non-viral delivery system with a gene under control of a promoter sequence, the gene capable of expressing at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein.

24. A stable cell line, comprising:
a gene under control of a promoter sequence, the gene capable of expressing at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein.

25. A method of manufacturing a composition for treating cancer, comprising:
providing a cell from a stable cell line capable of overexpressing at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein; and
growing the cell; and
extracting the overexpressed at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein.

26. The method according to claim 25, wherein the overexpression can be controlled via the introduction of doxycycline.

27. A method for ex vivo screening of cancers, comprising:
receiving a sample of a bodily fluid of a subject;
measuring a level of expression, in the subject, of at least the first 94 amino acids of a Tinagl1 protein, any fragments with conservative substitution showing 90% or greater homology to amino acids 22-94 of a Tinagl1 protein, or a signaling peptide fused or attached to a fragment with conservative substitution showing 90% or greater homology to amino acids 22-94 of a Tinagl1 protein;
determining whether the measured level of expression is below a predetermined threshold.

28. A method for ex vivo screening of cancers, comprising:
receiving a measurement of a level of expression, in a subject, of at least the first 94 amino acids of a Tinagl1 protein, any fragments with conservative substitution showing 90% or greater homology to the first 94 amino acids of a Tinagl1 protein, or a signaling peptide fused or attached to a fragment with conservative substitution showing 90% or greater homology to amino acids 22-94 of a Tinagl1 protein;
making a determination that the subject should be treated for cancer when the measured level of expression is below a predetermined threshold.
